(19) United States
(12) Patent Application Publication
(10) Pub. No.: US 2005/0267009 A1
(43) Pub. Date: Dec. 1, 2005

(54) Method of Treatment of Acute and Persistent Pain

(76) Inventor: William R. Deagle, Centennial, CO (US)

Correspondence Address: KYLE W. ROST, 5490 AUTUMN CT., GREENWOOD VILLAGE, CO, 80111, US

(21) Appl. No.: 10/908,867

(22) Filed: May 30, 2005

Related U.S. Application Data
(60) Provisional application No. 60/521,589, filed on May 28, 2004.

(51) Int. Cl. A61K 38/17; A61K 48/00

(52) U.S. Cl. 514/2; 514/44

(57) ABSTRACT

Biochemical mediators of inflammation facilitate persistent pain disorder. Treatment follows a multilevel pain gate model to enable site-specific application of appropriate inhibitors that block receptor sites. The result often is the effective treatment of the pain disorder by the administration of lower dosages of the inhibitors than used according to other treatment models.

### BACKGROUND OF INVENTION

1. Field of the Invention

This invention generally relates drug, bio-affecting and body treating compositions. More specifically, the invention relates to a method of treatment of persistent pain utilizing a model that will be referred to as “Deagle's Multilevel Pain Gate Molecular Law.” The novel result enabled by the model is that persistent pain is treated in a site-specific manner by inhibiting pain gate facilitator molecules.

2. Description of Prior Art

The current theories and treatment options for persistent pain are not satisfactory. The population of patients with chronic pain and disrupted lives grows constantly. According to the American Pain Foundation, there are 75 million Americans who have chronic pain. Pain is the second most common reason for doctor visits. Unless we can understand how pain is generated, we cannot provide a solution. Our understanding of pain has not advanced since the 1965 publication of the gate theory of pain by Canadian psychologist Ronald Meizack and British physiologist Patrick Wall. In their paper titled “Pain Mechanisms: A New Theory,” Meizack and Wall suggested a gating mechanism within the spinal cord that closed in response to normal stimulation of the fast conducting “touch” nerve fibers; but opened when the slow conducting “pain” fibers transmitted a high volume and intensity of sensory signals. The gate could be closed again if these signals were countered by renewed stimulation of the large fibers.

A recent model, known as Sota Omoigui's Law, proposes that the origin of all pain is inflammation and the inflammatory response. This model is a dramatic and revolutionary shift from a focus on structural pathology to an understanding of the biochemical origin of Pain. United States published patent application U.S. 2004/0038874 describes this model in greater detail.

Medical theories tend to place an over reliance on structural abnormalities to explain pain syndromes. This is not surprising because our current imaging technologies are structure based. Physicians are comfortable treating what they see. Patients who have structural abnormalities such as a herniated disk on MRI scans get operated upon often times needlessly and end up with more back or neck pain. Patients with severe pain who do not have structural abnormalities on MRI scans are dismissed as psychiatric cases. The fallacy of this approach has been confirmed in numerous published studies.

In one of these studies, the authors performed magnetic resonance imaging on sixty-seven individuals who had never had low-back pain, sciatica, or neurogenic claudication. The scans were interpreted independently by three neuro-radiologists who had no knowledge about the presence or absence of clinical symptoms in the subjects. About one-third of the subjects were found to have a substantial abnormality. Of those who were less than sixty years old, 20 percent had a herniated nucleus pulposus and one had spinal stenosis. In the group that was sixty years old or older, the findings were abnormal on about 57 percent of the scans: 36 percent of the subjects had a herniated nucleus pulposus and 21 percent had spinal stenosis. There was degeneration or bulging of a disc at least at one lumbar level in 35 percent of the subjects between twenty and thirty-nine years old and in all but one of the sixty to eighty-year-old subjects. In view of these findings in asymptomatic subjects, the authors concluded that abnormalities on magnetic resonance images must be strictly correlated with age and any clinical signs and symptoms before operative treatment is contemplated.

In another study, the authors examined the prevalence of abnormal findings on magnetic resonance imaging (MRI) scans of the lumbar spine in people without back pain. 52 percent of the asymptomatic subjects were found to have a bulge at least at one level, 27 percent had a protrusion, and 1 percent had an extrusion. Thirty-eight percent had an abnormality of more than one intervertebral disk. The prevalence of bulges, but not of protrusions, increased with age. The most common nonintervertebral disk abnormalities were Schmorl's nodes (herniation of the disk into the vertebral-body end plate) found in 19 percent of the subjects; annular defects (disruption of the outer fibrous ring of the disk) in 14 percent; and facet arthropathy (degenerative disease of the posterior articular processes of the vertebrae) in 8 percent. The findings were similar in men and women. The authors concluded that on MRI examination of the lumbar spine, many people without back pain have disk bulges or protrusions but not extrusions. The authors went further to state that given the high prevalence of these findings and of back pain, the discovery by MRI of bulges or protrusions in people with low back pain may frequently be coincidental.

In another study, which tracked the natural history of individuals with asymptomatic disc abnormalities in magnetic resonance imaging, the authors stated that the high rate of lumbar disc alterations recently detected in asymptomatic individuals by magnetic resonance imaging demands reconsideration of a pathomorphology-based explanation of low back pain and sciatica.

Chronic pain differs from acute pain in that it serves no useful function, causes suffering, limits activities of daily living, and increases costs of healthcare payments, disability, and litigation fees. Pain perception begins with activation of peripheral nociceptors and conduction through myelinated A delta and unmyelinated C fibers to the dorsal root ganglion. From here, signals travel via the spinothalamic tract to the thalamus and the somatosensory cortex. Modulation of sensory input (i.e., pain) occurs at many levels. Nociceptors are also neuroeffectors, and transmission can be modulated by their cell bodies, which secrete inflammatory mediators, neuropeptides, or other pain-producing substances. Descending pathways from the hypothalamus, which has opioid-sensitive receptors and is stimulated by arousal and emotional stress, can transmit signals to the dorsal horn that modulate ascending nociceptive transmissions. Modulation to alter the perception of pain also can occur at higher centers (e.g., frontal cortex, midbrain, medulla) by opioids, anti-inflammatory agents, as well as antagonists and agonists of neurotransmitters. Spinal cord cells called “glia” are also critically important. Indeed, when glia become activated, they begin releasing a variety of chemical substances that causes the pain message to become amplified, thus causing pain to hurt more.

A promising therapeutic target for the treatment and prevention of chronic pain and opioid tolerance/hyperalgesia is the modulation of the central nervous system (CNS) immunological response that ensues following injury or opioid administration. Broadly defined, central neuroimmune activation involves the activation of cells that interface with the peripheral nervous system and blood. Activation of these cells, as well as parenchymal microglia and astrocytes by injury, opioids, and other stressors, leads to subsequent production of cytokines, cellular adhesion molecules, chemokines, and the expression of surface antigens that enhance a CNS immune cascade. This response can lead to the production of numerous pain mediators that can sensitize and lower the threshold of neuronal firing: the pathologic correlate to central sensitization and chronic pain states. Recent advancements in the pain field have identified a central nervous system (CNS) neuroimmune response that may act as the driving force for neuronal hypersensitivity, the pathological correlate to chronic pain following peripheral nerve injury.

The physiology of nociception involves a complex interaction of peripheral and central nervous system (CNS) structures, extending from the skin, the viscera and the musculoskeletal tissues to the cerebral cortex. The pathophysiology of chronic pain shows alterations of normal physiological pathways, giving rise to hyperalgesia or allodynia. After integration in the spinal cord, nociceptive information is transferred to thalamic structures before it reaches the somatosensory cortex. Each of these levels of the CNS contain modulatory mechanisms. The two most important systems in modulating nociception and antinociception, the N-methyl-D-aspartate (NMDA) and opioid receptor system, show a close distribution pattern in nearly all CNS regions; and activation of NMDA receptors has been found to contribute to the hyperalgesia associated with nerve injury or inflammation. Apart from substance P (SP), the major facilitatory effect in nociception is exerted by glutamate as the natural activator of NMDA receptors. Stimulation of ionotropic NMDA receptors causes intraneuronal elevation of Ca2+, which stimulates nitric oxide synthase (NOS) and the production of nitric oxide (NO). NO as a gaseous molecule diffuses out from the neuron and, by action on guanylyl cyclase, NO stimulates in neighboring neurons the formation of cGMP. Depending on the expression of cGMP-controlled ion channels in target neurons, NO may act excitatory or inhibitory. NO has been implicated in the development of hyperexcitability, resulting in hyperalgesia or allodynia, by increasing nociceptive transmitters at their central terminals.

Among the three subtypes of opioid receptors, mu- and delta-receptors either inhibit or potentiate NMDA receptor-mediated events, while kappa opioids antagonize NMDA receptor-mediated activity. Recently, corticotrophin releasing hormone (CRH) has been found to act at all levels of the neuraxis to produce analgesia. Modulation of nociception occurs at all levels of the neuraxis, thus eliciting the multidimensional experience of pain involving sensory-discriminative, affective-motivational, cognitive and locomotor components.

Chronic pain can occur after peripheral nerve injury, infection, or inflammation. Under such neuropathic pain conditions, sensory processing in the affected body region becomes grossly abnormal. Despite decades of research, currently available drugs largely fail to control such pain. This invention explores the possibility that the reason for this failure lies in the fact that such drugs were designed to target neurons rather than immune or glial cells. Immune cells are a natural and inextricable part of skin, peripheral nerves, dorsal root ganglia, and spinal cord. Immune and glial activation may participate in the etiology and symptomatology of diverse pathological pain states in both humans and laboratory animals. Of the variety of substances released by activated immune and glial cells, proinflammatory cytokines (tumor necrosis factor, interleukin-1, interleukin-6) appear to be of special importance in the creation of peripheral nerve and neuronal hyperexcitability.

The pathology of chronic pain (neuropathic pain) differs from that of nociceptive pain, and conventional pharmacological treatment of chronic central pain is usually less successful than treatment of inflammation-related pain. The many newly discovered mechanisms for the transmission and modulation of pain impulses are characterized by complex activity-dependent plasticity, which means that therapeutic strategies for persistent pain must be adapted to changing targets—either at the site of injury or at other sites in the central nervous system.

Neuroimmune activation involves the activation of nonneuronal cells such as endothelial and glial cells, which when stimulated leads to enhanced production of a host of inflammatory mediators, such as cytokines. The central production of proinflammatory cytokines, such as interleukin-1 beta (IL-1 beta), IL-6 and tumor necrosis factor have been found to play a key role in the propagation of persistent pain states. In addition, chemotactic cytokines, chemokines, have also been recently identified in the CNS neuroimmune cascade that ensues after injury to a peripheral nerve. The extravasation of leukocytes from the blood to the site of perceived injury is defined as the neuroinflammatory aspect of this cascade. Chemokines directly control this leukocyte transmigration process. They are synthesized at the site of injury and establish a concentration gradient through which immune cells migrate.

Recent studies have demonstrated leukocyte trafficking into the CNS following peripheral nerve or lumbar nerve root injury. With the use of selective cytokine inhibitors and neutralizing antibodies, tactile and thermal hypersensitivity is attenuated in animal models of neuropathy. Cytokines produced by spinal cord glia after peripheral inflammation, infection or trauma have a relevant role in the maintenance of pain states. The effect of intrathecally administered interleukin-1 beta (IL-1 beta) on spinal cord nociceptive transmission was studied in normal and monoarthritic rats by assessing wind-up activity in a C-fiber-mediated reflex paradigm evoked by repetitive (1 Hz) electric stimulation. Low i.t. doses of IL-1 beta (0.03, 0.12, 0.5 and 2.0 ng) dose-dependently enhanced wind-up activity in normal rats, while higher doses (8.0 ng) only produced a marginal insignificant effect. IL-1 beta administration to monoarthritic rats did not significantly change wind-up scores at any dose. Adaptive changes developed in the spinal cord during chronic pain may underlie the ineffectiveness of exogenous IL-1 beta to up-regulate nociceptive transmission.

A study investigated whether interleukin-1 (IL-1), a mediator of inflammatory pain, also plays a role in pain induced by nerve injury. Female C57BL/6-mice with a chronic constrictive injury of one sciatic nerve, an established model of neurogenic hyperalgesia and allodynia, were treated with different doses (10-80 microg) of a neutralizing monoclonal rat antibody to IL-1 receptor I (anti-IL-1RI). This antibody dose-dependently reduced thermal hyperalgesia and mechanical allodynia in the animals. Furthermore, immunoreactivity for the proinflammatory cytokine tumor necrosis factor-alpha (TNF) was reduced in mice treated with the highest dose of anti-IL-1RI. Degeneration of myelinated fibers was not altered by any of the treatment schedules. It appears IL-1 may be a mediator of hyperalgesia after nerve lesion.

TNF-alpha expression of human Schwann cells may be up-regulated in painful neuropathies. The elevation of sTNF-RI in patients with centrally mediated mechanical allodynia suggests that systemic sTNF-RI levels may influence central pain processing mechanisms. Proinflammatory cytokines contribute to the regulation of the disease process in inflammatory neuropathies. Cellular localization of cytokine expression in CIDP nerve biopsies should provide further insight into the pathogenic mechanisms of the disease and the individual cells involved. In this study, in situ hybridization was used to determine the exact localization and identity of cells that express TNF alpha, IFN gamma and IL-2 mRNA within the CIDP nerve. Paraffin embedded and frozen sural nerve biopsies from three acute phase CIDP patients were used for the study. Sections of these samples were probed with digoxigenin labeled oligoprobes for TNF alpha, IFN gamma and IL-2. The results demonstrate localization of cytokine expression to the inner rim of the perineurium, epineurial and endoneurial blood vessels and infiltrating inflammatory cells. In addition strong staining for TNF alpha mRNA was widespread in the endoneurium in areas consistent with/suggestive of Schwann cells.

Expression of cytokines in the perineurium and endoneurial blood vessels may have pertinent implications with respect to the breakdown of the blood nerve barrier associated with CIDP. In the very least the potential for an immunomodulatory role may be ascribed to these cells. The NMDA receptor is upregulated in pathways in which the pain gate is in the open position at all levels of the seven level pain gate.

Another study considered the relationship between wind-up, temporal summation and central sensitization. In particular, the role of NMDA receptor mechanisms in the modulation of wind-up/temporal summation was discussed. The results indicate that the study of wind-up and temporal summation has given information about some of the complex mechanisms underlying central sensitization. Both wind-up and temporal summation appear to be dependent on NMDA receptor activation. The results of clinical trials in patients with chronic pain suggest that the NMDA receptor may represent a new target for modulation of abnormal temporal summation of pain, as well as other characteristics of chronic pain.

Experimental results show that agmatine inhibits the production of nitric oxide by decreasing the activity of NOS-2 in macrophages and astroglial cells by decreasing the levels of NOS-2 protein. These findings provide a molecular basis for the neuroprotective and anti-inflammatory actions of agmatine.

Skin mast cells are involved in inflammatory reactions during the CRPS1. Mast cells could play a role in the production of cytokines such as TNF-alpha.

Free radical superoxide anion is involved in the pain pathway in the spinal cord. Blockade or enzymatic or genetic manipulation of the production, capture or disposal or superoxide anion and other free radicals such as hydroxyl radical or peroxynitrous radical are essential to reduce free radical dependent pain gated pathways throughout the nervous system at all seven levels. Pain is thus a process of interplay between information going up each of the seven gates with inhibition at the gate and from signals inhibiting from above and below the gate at which pain signal is amplified. Thus to think just in terms of the inflammatory property of some of the pain gate facilitatory molecules or the inhibitory properties of the pain gate inhibitory molecules leaves out non-inflammatory molecules and neurotransmitters and many other molecules that modulate at one side or the other in different circumstances. Nitrous oxide is an excellent example of such a molecule which depending on the site of activation, the enzyme, and the ultimate molecular product, it is a neuromodulator to increase pain transmission, and not inflammatory. Nerve Growth Factors and other glial cell transforming peptides have similar neuromodulatory actions, but are not inflammatory to tissues, but increase pain gate facilitation of pain signal transmission.

In one study, at the level of the spinal cord, and at time of peak hyperalgesia, endogenous manganese superoxide dismutase was nitrated and subsequently deactivated, losing its capacity to remove superoxide. The anti-hyperalgesic effects of M40403 were not reversed by naloxone, excluding potential involvement of an opiate pathway. Collectively these studies have unraveled a critical role for superoxide in the nociceptive signaling cascade both peripherally and centrally. The discovery of this pathway opens a new therapeutic strategy for the development of novel non-narcotic anti-hyperalgesic agents.

Utilizing a rat model, with comparative DRG, dorsal root ganglionic proximal and distal L5 root level neurotraumatic models, levels of cytokines were measured. Spinal IL-1 beta, IL-6, IL-10, and TNF mRNA and IL-6 protein were significantly elevated in both injuries. The overall magnitude of expression and temporal patterns were similar in both models of injury.

Proinflammatory cytokines are supposed to play a major role in the pathophysiology of vasculitis and in the development of neuropathic pain. A study investigated the cytokine expression in sural nerve biopsy specimens from patients with vasculitic and other inflammatory and non-inflammatory neuropathies, and investigated whether an increased cytokine expression was correlated with the presence of neuropathic pain. Immunohistochemistry including double labeling and morphometry was used to localize and quantify the expression of interleukin-1 beta (IL-1 beta), IL-6, and tumor necrosis factor-alpha (TNF) in sural nerve biopsy samples of 41 patients with vasculitic neuropathy (VANP), chronic inflammatory demyelinating neuropathy (CIDP), non-inflammatory chronic axonal neuropathy (CANP), and 3 controls. Overall cytokine immunoreactivity was highest in VANP, less strong in CIDP, and lowest in CANP. Cytokine immunoreactivity was directly correlated with the degree of axonal degeneration, endoneurial macrophages and epineurial T cells. In VANP and CANP, a higher cytokine content was associated with neuropathic pain.

Molecules may be neuromodulatory and also have properties in other tissues and circumstances that are proinflammatory cytokines and may contribute to the development of inflammatory and neuropathic pain and hyperalgesia in many in vivo models. Non-inflammatory neuropathic pain results when just the neuromodulatory properties of pain gate facilatory molecules increase signal transmission, without tissue inflammation. The primary pain disorder is therefore not dependent on inflammation but may accompany it in many clinical conditions, as these molecules that open the pain gate are often pro-inflammatory, but not always.

The rat skin model was used to investigate the effects of proinflammatory cytokines on the basal and heat-evoked release of calcitonin gene-related peptide from nociceptors in vitro. In contrast to the excitatory effects of cytokines observed in vivo, none of the cytokines tested evoked any calcitonin gene-related peptide (CGRP) release at normal skin temperature of 32 degrees C. However, the cytokines IL-1 beta, tumor necrosis factor (TNF)-alpha, and IL-6, but not IL-8, induced a pronounced and transient sensitization of the heat-evoked CGRP release from nociceptors in vitro. This heat sensitization was dose dependent, with EC(50) for IL-1 beta of 2.7 ng/ml and for TNF-alpha of 3.1 ng/ml. The maximum IL-1 beta effect reached almost 600% of the heat-evoked release, and the maximum TNF-alpha effect induced a rise in CGRP release of 350%. In contrast to IL-1 beta and TNF-alpha, IL-6 did not induce heat sensitization when applied alone but was only effective in the presence of soluble IL-6 receptor. This suggests a constitutive expression of signaling receptors for TNF and IL-1 beta and the signal transduction molecule gp130 but not IL-6 receptor or IL-8 receptor.

Furthermore, the acute cytokine signaling observed in this study was independent of transcriptional pathways because sensitization occurred on short latency in vitro and under conditions that excluded chemotactic accumulation of immune cells from blood vessels. The results demonstrate that interleukins may play an important role in the initiation of heat hyperalgesia in inflammation and neuropathy.

Mechanical allodynia, but not thermal hyperalgesia induced by intraneural TNF, was reduced by ibuprofen, but not by celecoxib treatment 5 and 7 days after injection. Sciatic nerves, lumbar dorsal root ganglia (DRG) and spinal cords from rats with treatment started 12 hours after surgery were analyzed for prostaglandin E2 (PGE2) levels 10 days after CCI. In injured nerves and ipsilateral DRG, PGE2 levels were increased. Ibuprofen treatment reversed PGE2 levels in injured nerves and DRG, whereas celecoxib blocked increased PGE2 levels only in nerves. In spinal cord, no change in PGE2 levels was observed. In contrast to the marked inhibition of nerve-injury-induced upregulation of PGE2 by COX inhibitors, the effect on pain behavior was modest.

Cytokines orchestrate T cell-mediated immune responses. In experimental autoimmune encephalomyelitis (EAE) the proinflammatory cytokines interferon (IFN)-gamma, tumor necrosis factor (TNF)-alpha, interleukin (IL)-1 beta, IL-6, IL-12 and IL-18 are critically involved in the initiation and amplification of the local immune response in the CNS, which is counter-balanced by upregulation of antiinflammatory cytokines such as IL-10. Monotherapy with antibodies to tumor necrosis factor-alpha (TNF) or interleukin-1 receptor 1 (IL-1R1) reduces hyperalgesia in an animal model of painful neuropathy. A study investigated whether combined therapy with epineurial anti-TNF and anti-IL-1R1 antibodies produces a further advantage. C57BL/6 mice with a chronic constrictive injury of one sciatic nerve were treated epineurially with neutralizing antibodies to either IL-1R1 or TNF alone or with a combined application of neutralizing antibodies to TNF and IL-1R1. Combined treatment with anti-IL-1R1 and anti-TNF antibodies markedly reduced thermal hyperalgesia and mechanical allodynia more effectively than monotherapy with either antibody. There were no detectable differences in IL-1 beta and TNF endoneurial protein expression between animals with monotherapy and combined treatment. This study concludes that combined anti-cytokine therapy may be a useful strategy in the treatment of neuropathic pain.

Exogenous spinal IL-6 (100-500 ng) had no significant effect on electrically evoked neuronal responses in naive rats. In contrast, following neuropathy, spinal IL-6 produced a dose-related inhibition of the electrically evoked C-fibre, initial C-fibre and measures of neuronal hyperexcitability (post discharge and wind-up). In addition, spinal IL-6 markedly inhibited mechanical neuronal responses in neuropathic rats. Higher doses of spinal IL-6 also inhibited, to a lesser degree, the initial C-fibre, post discharge and wind-up responses in sham-operated rats. These studies show that following nerve injury the actions of the cytokine alter so that spinal administration of IL-6 elicits anti-nociceptive effects not observed under normal conditions. Moreover, the inhibitory effects of IL-6 on C-fibre activity and neuronal hyperexcitability, suggest IL-6 to be a potential modulator of neuropathic pain.

In the rat neuropathy model, low dose (0.01-0.001 microg) goat anti-rat IL-6 i.t. administration (P=0.025) significantly decreased allodynia and trended towards significance at the higher dose (0.08 microg to 0.008 microg, P=0.062). Low doses (0.01-0.001 microg) i.t. normal goat and rat IgG significantly attenuated mechanical allodynia, but not at higher doses (0.08-0.008 microg; P=0.001 for both goat and rat IgG). These data provide further evidence for the role of central IL-6 and neuroimmune modulation in the etiology of mechanical allodynia following peripheral nerve injury.

VR1 receptors, present on Adelta- and C-fibres and post-synaptic sites within the spinal cord dorsal horn, are an integrator of noxious stimuli. Here, the contribution of spinal VR1 receptors to spinal nociceptive processing in nerve injured (selective spinal nerve ligated SNL) and sham anaesthetised rats was studied. Spinal capsazepine (0.5-30 microM), a competitive VR1 antagonist, reduced noxious evoked responses of spinal neurones to a greater extent in sham operated rats, compared to SNL rats. Significant differences between the effect of spinal capsazepine on the non-potentiated component of the C-fibre evoked response of SNL and sham operated rats are reported (p<0.01, two-way ANOVA). Data suggests there is a functional plasticity of the spinal VR1 receptor following nerve injury.

An investigation of the effect of a potent cannabinoid agonist, HU210, on somatosensory transmission, was performed in a model of neuropathic pain. Spinal administration of HU210 (0.5-500 ng/50 microl) significantly reduced the C-fibre mediated post-discharge response of spinal neurones in sham operated, but not nerve injured rats. By contrast, spinal HU210 significantly reduced Adelta-fibre evoked responses of spinal neurones in both sham operated and nerve injured rats. Systemic administration of HU210 (6-60 microg/kg) significantly reduced C- and Adelta-fibre evoked responses of spinal neurones in sham-operated rats. HU210 (60 microg/kg) inhibited the overall C-fibre evoked response (54+/−8% of control, p<0.01), post-discharge response (28+/−12% of control, p<0.01), and Adelta-fibre evoked (48+/−5% of control p<0.01) responses of spinal neurones. In nerve injured rats, systemic administration of HU210 did not significantly reduce C- or Abeta-fibre evoked responses of spinal neurones. This study demonstrates plasticity of the spinal cannabinoid receptor system following peripheral nerve injury. Cannabinoid receptors throughout the neuraxis reduce nerve firing and hence pain signal transduction. Molecular modifications to future engineered cannabinoind neuropharmaceuticals may be yet more powerful at reducing pain in chronic neuropathic pain.

Thalidomide has several targets and mechanisms of action: a hypnosedative effect, several immunomodulatory properties with an effect on the production of TNF-alpha and the balance between the different lymphocyte subsets and an antiangiogenic action. Thalidomide reduces thermal hyperalgesia and mechanical allodynia in chronic constrictive sciatic nerve injury (CCI). Since thalidomide mainly inhibits tumor necrosis factor alpha (TNF-alpha) synthesis with less well defined effects on other cytokines, researchers have investigated the effect of the drug on the expression of the proinflammatory cytokines TNF-alpha, interleukin-1 beta (IL-1 beta) and interleukin 6 (IL-6), and of the anti-inflammatory cytokine interleukin-10 (IL-10) in the lesioned rat sciatic nerve. The increase of endoneurial TNF-alpha during the first week after CCI was reduced after thalidomide treatment, as shown with immunohistochemistry and enzyme-linked-immunosorbent assay. In contrast, endoneurial IL-1 beta-immunoreactivity (IR) and IL-6-IR were not altered by thalidomide treatment, nor was macrophage influx. Pain gate Inhibitory Molecular increases with thalidomide has profound effects on blocking the pain gate. Recruitment of epineurial IL-10 immunoreactive macrophages as well as the recovery of injury-induced depletion of endoneurial IL-10-IR was enhanced by thalidomide treatment. To control for central plasticity as another factor for the effects of thalidomide, the spinal cord was analyzed for changes in neurotransmitters. The decrease in CGRP-IR and SP-IR in the dorsal horn of operated animals was not influenced by treatment. In contrast, the increase in met-enkephalin observed in the dorsal horn of operated animals was further enhanced in the thalidomide-treated animals. The study elucidates some of the complex alterations in CCI and its modulation by thalidomide, and provides further evidence for a possible therapeutic benefit of cytokine-modulating substances in the treatment of neuropathic pain.

Both ipsilateral and mirror-image SIN-induced allodynias are (1) reversed by intrathecal (peri-spinal) delivery of fluorocitrate, a glial metabolic inhibitor; (2) prevented and reversed by intrathecal CNI-1493, an inhibitor of p38 mitogen-activated kinases implicated in proinflammatory cytokine production and signaling; and (3) prevented or reversed by intrathecal proinflammatory cytokine antagonists specific for interleukin-1, tumor necrosis factor, or interleukin-6. Reversal of ipsilateral and mirror-image allodynias was rapid and complete even when SIN was maintained constantly for 2 weeks before proinflammatory cytokine antagonist administration. These results provide the first evidence that ipsilateral and mirror-image inflammatory neuropathy pain are created both acutely and chronically through glial and proinflammatory cytokine actions.

Animal studies have proven with TNF-Alpha and IL-1 that proinflammatory cytokines are involved not only in inflammatory pain but also in neuropathic pain.

Propentofylline has the ability to inhibit glial activation and enhanced spinal proinflammatory cytokines following peripheral nerve injury. This finding along with earlier observations of an anti-allodynic activity of propentofylline using the identical animal model of mononeuropathy supports the concept that modulation of glial and neuroimmune activation may be potential therapeutic targets to treat or prevent neuropathic pain. Non-inflammatory action to block glial cell activation, and thus act as pain gate inhibitory molecules is the primary operational molecular target for propentofylline. Further, restoration of the analgesic activity of morphine by propentofylline treatment suggests that increased glial activity and proinflammatory cytokine responses may account for the decreased analgesic efficacy of morphine observed in the treatment of neuropathic pain.

Following nerve injury there is a novel mexiletine sensitive spinal substrate which contributes to Adelta-fibre and C-fibre, but not Abeta-fibre, somatosensory transmission. This central action may underlie some of the clinical efficacy of mexiletine in the treatment of neuropathic pain states. Again consistent with “Deagle's Multilevel Pain Gate Model,” this pharmaceutical is not anti-inflammatory, but blocks as a pain gate inhibitory molecule, the pain transmission from one or more levels of the gating neurological hierarchical control network.

The anti-apoptosis activities of Erythropoitin, Epo/EpoR/JAK2 in DRG neurons is capable of blocking protracted pain states with reductions in pain behaviours, and therefore support a role for Epo therapy in the treatment of neuropathic pain. Thus again, this molecule operates as a pain gate inhibitory and not an anti-inflammatory mechanism.

A recent PET study revealed that the first and second somatosensory cortices (SI, SII), and the anterior cingulate cortex are activated by painful peripheral stimulation in humans. It has become clear that painful signals (nociceptive information) evoked at the periphery are transmitted via various circuits to the multiple cerebral cortices where pain signals are processed and perceived. Human or clinical pain is not merely a modality of somatic sensation, but associated with the affect that accompanies sensation. Consequently, pain has a somatosensory-discriminative aspect and an affective-cognitive aspect that are processed in different but correlated brain structures in the ascending circuits. Considering the physiologic characteristics and fiber connections, the SI and SII cortices appear to be involved in somatosensory-discriminative pain, and the anterior cingulate cortex (area 24) in the affective-cognitive aspect of pain.

The seventh level, anterior cortical pain gate has been demonstrated in mice. In the new study, Dr. Zhuo and his team tested pain-related behavior in normal (control) mice and in mice missing the gene for two proteins called adenylyl cyclase 1 and 8 (AC1 and AC8). These two enzymes are found primarily in a part of the forebrain called the anterior cingulate cortex (ACC). Previous studies have shown that this region is important for feeling pain. The two groups of mice reacted the same way to stimuli that cause acute pain. However, in tests of chronic pain, the mice without AC1 and AC8 had much smaller reactions than the control mice, suggesting that they did not feel as much pain. When the researchers gave these mice a substance that increases the amount of a chemical called cyclic AMP, which triggers changes in neuronal connections, they began to react to painful stimuli like the normal mice did. This showed that disabling the two genes blocked chronic pain by preventing pain-related changes in neuronal connections, rather than by permanently altering the brain during development. Gene modulation alters the cortical pain gate as inhibitory pain gate molecules, and does not operate as anti-inflammatory molecules.

Fibromyalgia has peripheral skin abnormalities of cytokines in 30% of patients of TNF-Alpha and IL1-Beta. As per Deagle's Law of the Multilevel Pain Gating, changes at one level or gate will affect cytokines, genes, and facilitating neurotransmitters and receptors such as NMDA receptor density. Thus one can expect to find both PET scan cortical and subcortical thalamic nuclei metabolism, and quantitative sensory nerve conduction threshold testing abnormalities, as well as measurable skin Langerhans complex cytokine and pro-faciliatatory neurotransmitter levels.

In one study, abnormal temporal summation of second pain (wind-up) and central sensitization have been described recently in patients with FMS. Wind-up and central sensitization, which rely on central pain mechanisms, occur after prolonged C-nociceptor input and depend on activation of nociceptor-specific neurons and wide dynamic range neurons in the dorsal horn of the spinal cord. Other abnormal central pain mechanisms recently detected in patients with FMS include diffuse noxious inhibitory controls. These pain inhibitory mechanisms rely on spinal cord and supraspinal systems involving pain facilitatory and pain inhibitory pathways. Brain-imaging techniques that can detect neuronal activation after nociceptive stimuli have provided additional evidence for abnormal central pain mechanisms in FMS. Brain images have corroborated the augmented reported pain experience of patients with fibromyalgia during experimental pain stimuli. In addition, thalamic activity, which contributes significantly to pain processing, was decreased in fibromyalgia. However, central pain mechanisms of fibromyalgia may not depend exclusively on neuronal activation. Neuroglial activation has been found to play an important role in the induction and maintenance of chronic pain.

Level seven anterior cortical to deeper nuclear pain control gating is further demonstrated by a pain-signaling pathway in the brain, modulated by the neurotransmitter GABAB, which could be the source of a potential therapy for controlling chronic pain, according to a recent study partly funded by the National Institute of Arthritis and Musculoskeletal and Skin Diseases (NIAMS).

The study by Luc Jasmin, M.D., of the University of California, San Francisco and his colleagues from Harvard Medical School and Catholic University (Milan, Italy), looked at two pain-signaling pathways in animal models: communication between the RAIC (rostral agranular insular cortex, a small region of the cerebral cortex) and the amygdala (a brain area involved in pain, fear and attention), and between the RAIC and the locus coeruleus (an area involved in inhibiting pain signals from the brain to the rest of the body). Their work showed that signals from the RAIC to the amygdala—inhibited by GABAB—might be involved in chronic pain. When the RAIC sends signals to the amygdala, pain is experienced; equally, communication between the RAIC and the locus coeruleus increases pain. Therefore, increasing GABAB in the RAIC silences the pathway to the amygdala or the locus and decreases pain. Making matters more complicated, both signaling pathways act independently, and the one to the amygdala predominates. One can first decrease pain by blocking the pathway to the locus coeruleus and then increase pain by activating the pathway to the amygdala. Anesthetizing the amygdala then restores the initial condition of pain inhibition.

N-methyl-D-asparate (NMDA) receptors activation and the subsequent nitric oxide (NO) production probably play a between-systems mechanism of opioid tolerance. Some recent studies also found that cyclooxygenase (COX) inhibitors could attenuate the opioid tolerance without enhancing morphine's antinociceptive effect. In the search for new therapeutic strategies, the gene-based approaches are of potential interest as they offer the possibility to introduce a therapeutic protein into some relevant structures and to drive its continuous production in the near vicinity of targeted cells. Recently, these techniques have been experimented in several animal models of chronic pain, showing that transfer at the spinal level of some genes, in particular those of opioid precursors proopiomelanocortin or proenkephalin A, leading to the overproduction of products that they encode, attenuated persistent pain of both inflammatory and neuropathic origin. Thus, in polyarthritic rat, a model of chronic inflammatory pain, it was demonstrated that herpes simplex virus vector mediated overexpression of proenkephalin A in primary sensory neurons at the lumbar level elicited both antihyperalgesic and anti-inflammatory activities. Apart from opioids, numerous other molecules involved in pain processing are of potential therapeutic interest for gene-based protocols. For instance, targeting some molecules involved in pain induction and perpetuation, such as proinflammatory cytokines, raises an interesting possibility to block the “development” of pain. The clinical application of these approaches remains to be established, and, presently, one of the main problems to be solved is the innocuity of virus-derived vectors. However, the experimental use of gene-based techniques might be particularly useful for the evaluation of the therapeutic interest of some recently identified molecules involved in pain processing and might finally lead to the development of new “classical” pharmacological tools.

Gene transfer to various levels of the CNS at any of the seven pain gate levels has the potential to block pain transduction and transmission. Again, pain gate inhibitory molecular mechanisms were operational consistent with “Deagle's Multilevel Pain Gate Model.” The first experimental protocols attempted the transfer of opioid precursor genes and their overexpression mainly at the spinal level. They demonstrated the feasibility and the real interest of these approaches by showing that local overproduction of opioid peptides induced antinociceptive effects in animal models of persistent pain, of inflammatory-, neuropathic- and even cancerous origin. Future gene-based protocols will certainly target some of the recently identified molecules involved in pain transduction mechanisms, sensory nerve sensitization or pain perpetuation. The differential expression of neurotrophic factors and their cognate soluble receptors in human vasculitic neuropathy suggests that NGF, which was effectively transferred to sensory axons with p75, may induce pain.

The dorsal horn of the spinal cord represents an attractive site for interventions designed to treat chronic pain, but it has been difficult to identify small molecules that act selectively on pain transmission at the spinal level. One approach is the use of viral vector-mediated gene transfer to achieve focal production and release of short-lived analgesic peptides. Herpes simplex virus based vectors, expressing proenkephalin delivered by subcutaneous inoculation, transduce neurons of the dorsal root ganglion, leading to release of enkephalin from nerve terminals in dorsal horn to produce an analgesic effect in several models of chronic pain. Gene transfer to the DRG has thus been demonstrated to block pain transmission at this pain gate level.

Researchers in Britain in 1995 found the P2X3 receptor was expressed at high levels in nociceptive sensory neurones, consistent with earlier reports that ATP induced pain in humans and animals. At first it was thought that ATP was most likely to play a role in acute pain, following its release from damaged or stressed cells and since then a wide variety of experimental techniques and approaches have been used to study this possibility. Exogenous and endogenous ATP can indeed acutely stimulate sensory neurones, more recent reports using gene knockout and antisense oligonucleotide technologies, and a novel, selective P2X3 antagonist, A-317491, all indicate that ATP and P2X3 receptors are more likely to be involved in chronic pain conditions, particularly chronic inflammatory and neuropathic pain. These reports indicate that P2X3 receptors on sensory nerves may be tonically activated by ATP released from nearby damaged or stressed cells, or perhaps from the sensory nerves themselves. This signal, when transmitted to the CNS, will be perceived consciously as chronic pain. In addition, it is now clear that several subtypes of P2Y receptor are also expressed in sensory neurones. Although their distribution and functions have not been as widely studied as P2X receptors, the effects that they mediate indicate that they might also be considered as therapeutic targets in the treatment of pain.

A study analyzed the effect of intrathecal delivery of human IL-2 gene on rat chronic neuropathic pain induced by chronic constriction injury of the sciatic nerve. Human IL-2 cDNA was cloned into pcDNA3 containing a cytomegalovirus promoter. The paw-withdrawal latency induced by radiant heat was used to measure the pain threshold. The results showed that recombinant human IL-2 had a dose-dependent antinociceptive effect, but that this only lasted for 10-25 min. The pcDNA3-IL-2 or pcDNA3-IL-2/lipofectamine complex in contrast also showed dose-dependent antinociceptive effects, but these reached a peak at day 2-3 and were maintained for up to 6 days. Liposome-mediated pcDNA3-IL-2 produced a more powerful antinociceptive effect than pcDNA3-IL-2 alone. The paw-withdrawal latencies were not affected by control treatments such as vehicle, lipofectamine, pcDNA3, or pcDNA3-lipofectamine. In the experimental groups, human IL-2 mRNA was detected by reverse transcription-polymerase chain reaction in the lumbar spinal pia mater, dorsal root ganglion, sciatic nerve, and spinal dorsal horn, but not in gastrocnemius muscle. The expressed IL-2 profile detected by western blot coincided with its mRNA profile except it was present in the spinal dorsal horn at a higher level. Furthermore, human IL-2 assayed by enzyme-linked immunosorbent assay in cerebrospinal fluid could still be detected at day 6, but lower than day 3. The antinociceptive effect of pcDNA3-IL-2 could be blocked by naloxone, showing some relationship of the antinociceptive effect produced by IL-2 gene to the opioid receptors. It is hoped that the new delivery approach of a single intrathecal injection of the IL-2 gene described here may be of some practical use as a part of a gene therapy for treating neuropathic pain.

The biochemical mediators produced by the immune and glial cells include prostaglandins, leukotrienes, nitric oxide, TNF-Alpha, tumor necrosis factor alpha, IL-1, interleukin 1-alpha, IL1-Beta, interleukin 1-beta, IL-4, interleukin-4, IL6, Interleukin-6 and Il-8, interleukin-8, histamine, and serotonin.

Pain may also be associated with tissue inflammation, and local release of substances modulates local pain gate and nervous function allowing more signal transmission. The biochemical mediators produced by the nerve cells include inflammatory protein Substance P, kinins, calcitonin gene-related peptide (CGRP) neurokinin A and vasoactive intestinal peptide, and in the cortical neurons adenylyl cyclase proteins. Cell enzymes that catalyze reaction pathways and generate these biochemical mediators of inflammation include cyclooxygenase (COX) and lipoxygenase (LOX). A cell enzyme that is activated by inflammatory mediators such as TNF-alpha and interleukin-1 is Gelatinase B or matrix metallo-proteinase 9 (MMP-9). Once activated MMP-9 helps immune cells migrate through the blood vessels to inflammatory sites or to metastatic sites.

Drugs and medications which inhibit these biochemical mediators of inflammation include: non-steroidal anti-inflammatories, such as aspirin, tolmetin sodium, indomethacin and ibuprofen, inhibit the enzyme cyclooxygenase and therefore decrease prostaglandin synthesis. Prostaglandins are inflammatory mediators that are released during allergic and inflammatory processes. Phospholipase A2 enzyme, which is present in cell membranes, is stimulated or activated by tissue injury or microbial products. Activation of phospholipase A2 causes the release of arachidonic acid from the cell membrane phospholipid. From here there are two reaction pathways that are catalyzed by the enzymes cyclooxygenase and lipoxygenase. The cyclooxygenase enzyme pathway results in the formation of inflammatory mediator prostaglandins and thromboxane. Glucocorticoids are naturally occurring hormones that prevent or suppress inflammation and immune responses when administered at pharmacological doses. The anti-inflammatory corticosteroids inhibit the activation of phospholipase A.sub.2 by causing the synthesis of an inhibitory protein called lipocortin. It is lipocortin that inhibits the activity of phospholipases and therefore limits the production of potent mediators of inflammation such as prostaglandins and leukotriene.

Botulinum toxins are potent neurotoxins which block the release of neurotransmitters. One of these transmitters called acetylcholine is released by nerve cells and transported into muscle cells to signal the muscle to contract. Blockade of this transmitter by Botulinum toxin can produce a long lasting relief of muscle spasms. Botulinum toxins also inhibit the release of tumor necrosis factor alpha (TNF-alpha) from immune cells and thus can alleviate pain and spasm produced by the inflammatory response.

Tumor necrosis factor alpha-blocker medications have a central role in inflammatory responses. They have Interleukin-1 and TNF-alpha, because the administration of their antagonists, such as IL-1ra (Interleukin-1 receptor antagonist), soluble fragment of Interleukin-1 receptor, or monoclonal antibodies to TNF-alpha and soluble TNF receptor, all block various acute and chronic responses in animal models of inflammatory diseases. Etanercept (ENBREL) is a fusion protein produced by recombinant DNA technology. Etanercept binds to and inactivates Tumor Necrosis Factor (TNF-alpha) but does not affect TNF-alpha production or serum levels. Etanercept may also modulate other biologic responses that are induced or regulated by TNF-alpha such as production of adhesion molecules, other inflammatory cytokines and matrix metalloproteinase-3 (MMP-3 or stromelysin).

Infliximab is a monoclonal antibody targeted against tumor necrosis factor-alpha (TNF-alpha). Infliximab neutralizes the biological activity of the cytokine tumor necrosis factor-alpha (TNF-alpha). Infliximab binds to high affinity soluble and transmembrane forms of TNF-alpha and inhibits the binding of TNF-alpha with its receptors. Infliximab does not neutralize TNF-beta, a related cytokine that utilizes the same receptors as TNF-alpha. Biological activities attributed to TNF-alpha include induction of pro-inflammatory cytokines such as interleukin (IL)-1 and IL-6; enhancement of leukocyte migration by increasing endothelial layer permeability; expression of adhesion molecules by endothelial cells and leukocytes; activation of neutrophil and eosinophil functional activity; fibroblast proliferation; synthesis of prostaglandins; and induction of acute phase and other liver proteins.

Anakinra is a form of the human interleukin-1 receptor antagonist (IL-1Ra) produced by recombinant DNA technology. Anakinra differs from the naturally occurring native human IL-1Ra in that it has an additional methionine residue at its amino terminus. Anakinra acts similarly to the naturally occurring interleukin-1 receptor antagonist (IL-1Ra). IL-1Ra blocks effects of Interleukin-1 by competitively inhibiting binding of this cytokine, specifically IL-alpha and IL-beta, to the interleukin-1 type I receptor (IL-1R1), which is produced in a wide variety of tissues. IL-1Ra is part of the feedback loop that is designed to balance the effects of inflammatory cytokines.

Leflunomide interferes with RNA and protein synthesis in immune T and B-lymphocytes. T and B cell collaborative actions are interrupted and antibody production is suppressed. Leflunomide is the first agent for rheumatoid arthritis that is indicated for both symptomatic improvement and retardation of structural joint damage. Leflunomide may also have anti-inflammatory properties secondary to reduction of histamine release, and inhibition of induction of cyclooxygenase-2 enzyme (COX-2). Leflunomide may decrease proliferation, aggregation and adhesion of peripheral and joint fluid mononuclear cells. Decrease in the activity of immune lymphocytes leads to reduced cytokine and antibody-mediated destruction of joints and attenuation of the inflammatory process.

Phosphodiesterase inhibitors such as Pentoxifylline have other unique effects. The drugs suppress inflammatory cytokine production by T cells and macrophages. Some of the anti-inflammatory effect occurs by blocking nitric oxide (NO) production by macrophages. Pentoxifylline also blocks the production of Tumor Necrosis Factor Alpha. In one study, Pentoxifylline prevented nerve root injury and swelling (dorsal root ganglion compartment syndrome) caused by topical application of disk tissue (nucleus pulposus).

Tetracyclines such as doxycycline and minocycline may block a number of cytokines including interleukin-1, IFNg, NO-synthetases, and metalloproteinases. Interleukin-1 and IFN-gamma act synergistically with TNF-alpha and are known to be toxic to nerve tissue. 5-HT3-receptor antagonist medications such as Ondansetron diminish serotonin-induced release of substance P from C-fibers and prevent unmasking of NK2-receptors in the presence of serotonin.

Bisphosphonates medications such as Pamidronate reduce bone complications and related pain in patients with Paget's disease, osteoporosis and bone metastasis, thereby improving quality of life. Bisphosphonates have intrinsic anti-tumor activity by virtue of inducing tumor cell adherence to marrow, reducing interleukin-6 secretion, inducing tumor cell apoptosis, or inhibiting angiogenesis. Anti-depressant medication such as Amitriptyline also have effects on inflammatory mediators. Prolonged administration of amitriptyline and desipramine has resulted in a significant increase in the secretion of the anti-inflammatory cytokine interleukin-10. Anti-seizure medications such as Oxcarbazepine or Zonisamide decrease pain by reducing the rate of continuing discharge of injured and inflamed nerve fibers. Blockade of sodium channels in nerve cells leads to a decrease in electrical activity and a subsequent reduction in release of the excitatory nerve transmitter glutamate. Anti-seizure drugs also inhibit the initiation and propagation of painful nerve impulses by inhibiting nitric oxide synthetase activity. Nitric oxide synthetase is the enzyme responsible for the production of the inflammatory mediator nitric oxide. Anti-seizure drugs may also protect nerve cells from free radical damage by nitric oxide and/or hydroxyl radicals (OH−). Thalidomide and analogues mainly inhibit tumor necrosis factor alpha (TNF-alpha) synthesis but the drugs also have effects on other cytokines. Thalidomides increase the production of the anti-inflammatory cytokine interleukin-10 (IL-10) in lesioned sciatic nerves. In addition, thalidomides stimulate the release of the pain relieving natural opioid peptide methionine-enkephalin in the dorsal horn of the spinal cord.

### SUMMARY OF INVENTION

The invention is a method of treating persistent paid by closing the multilevel pain gate, utilizing multiple pharmaceutical, genopharmaceuticals and immunopharmaceutical technologies. These include but are not limited to, blocking genes with antisense-RNA or other methodologies such as nanotechnologies to block gene control for pain gate facilatory molecules (PGFM) that have only these properties or those that are both PGFM and inflammatory molecules, by blocking the cytokine receptors non-reversibly with immunopharmaceuticals that block receptors for cytokines with monoclonal antibodies, by soluble receptor blockade, supporting glial cell transformational blockade, and gene manipulation to down-regulate cytokine transcription or release. This invention covers use of the above range of pharmaceutical methodologies to increase pain gate inhibitory molecules (PGIM), and those molecules that are PGIM and also have anti-inflammatory properties. This invention broadly includes all molecules both modulatory and affecting increases and decreases in inflammation. The biochemical mediator of inflammation may be TNF-alpha; interleukin-1, leukotriene; 5-lipoxygenase; nitric oxide; substance P; calcitonin gene-related peptide; vasoactive intestinal peptide; interleukin-4; interleukin-6; interleukin-8; a kinin; serotonin; matrix metallo-proteinase; iNO synthase, inducible nitric oxide synthase and/or byproduct NO−, nitric oxide free radical; glial cell molecular mediator transformation activation, or combinations thereof.

Respectively, a suitable inhibitor of each biochemical mediator is a TNF-alpha inhibitor, an interleukin-1 receptor antagonist, a leukotriene receptor antagonist, a 5-lipoxygenase antagonist; a nitric oxide antagonist; a substance P antagoist; a calcitonin gene-related peptide antagonist; vasoactive intestinal peptide antagonist; an interleukin-4 antagonist; an interleukin-6 antagonist; an interleukin-8 antagonist, a kinin antagonist, a serotonin receptor antagonist; a matrix metallo-proteinase antagonist; an nitric oxide synthase antagonist; a glial cell mediator antagonist; a gene pain gate facilatory molecular inhibitor; and a gene pain gate inhibitory molecular upregulator. A persistent pain disorder may be Langerhans cellular junctional complex origin in the skin at the primary pain gates; the dorsal root ganglionic level or secondary pain gates; the substantia gelatinosa or tertiary pain gates; the ascending spinothalamic tracts or quaternary gates; the subthalamic and thalamic nuclei or fifth level pain gates; the sensory receptive-motor parietal-temporal cortical or sixth level gates; or the sensory-behavioral-motor frontal cortical or seventh level gates, or combinations thereof. The TNF-alpha inhibitor is administered intramuscularly, intravenously, by intra-articular injection, subcutaneously, orally, or rectally. TNF-alpha inhibitor is administered intravenously by injection or infusion wherein said dosage level is in the range of 2.5 mg/kg to 20 mg/kg. The TNF-alpha inhibitor is administered intramuscularly wherein said dosage level is in the range of 25 mg to 100 mg. The TNF-alpha inhibitor is administered orally at a dosage of about 20 mg to about 1,500 mg. The TNF-.alpha inhibitor is administered subcutaneously wherein said dosage level is in the range of 5 mg to 50 mg for acute or chronic regimens. The TNF-alpha inhibitor is administered by intra-articular injection wherein said dosage level is in the range of 25 mg to 100 mg. The TNF-alpha inhibitor is administered intranasally wherein said dosage level is in the range of 0.1 mg to 10 mg for acute or chronic regimens.

The TNF-alpha inhibitor is selected from the group consisting of etanercept, infliximab, CDP571 (a humanized monoclonal anti-TNF-alpha antibody), pegylated soluble TNF receptor Type I (PEGsTNF-R1), D2E7, thalidomide based compounds, Pentoxifylline and Phosphodiesterase inhibitors.

The interleukin-1 receptor antagonist is administered systemically or locally; parenterally; intramuscularly; intravenously; by intra-articular injection; subcutaneously; orally; or rectally. The interleukin-1 receptor antagonist is administered intravenously by injection or infusion wherein said dosage level is in the range of 2.5 mg/kg to 20 mg/kg. The interleukin-1 receptor antagonist is administered intramuscularly wherein the dosage level is in the range of 25 mg to 100 mg. The interleukin-1 receptor antagonist is administered orally at a dosage of about 20 mg to about 1,500 mg. The interleukin-1 receptor antagonist is administered subcutaneously wherein the dosage level is in the range of 5 mg to 50 mg for acute or chronic regimens. The interleukin-1 receptor antagonist is administered by intra-articular injection wherein the dosage level is in the range of 25 mg to 100 mg. The interleukin-1 receptor antagonist is administered intranasally wherein said dosage level is in the range of 0.1 mg to 10 mg for acute or chronic regimens. The interleukin-1 receptor antagonist is selected from the group consisting of naturally occurring and human recombinant interleukin-1 receptor antagonist. The leukotriene receptor antagonist is administered intramuscularly, intravenously, by intra-articular injection, subcutaneously, orally, or rectally.

The 5-lipoxygenase antagonist is administered intramuscularly, intravenously, by intra-articular injection, subcutaneously, orally, or rectally. The nitric oxide antagonist is selected from the group including Oxcarbazepine, Carbamazepine and Zonisamide. The nitric oxide antagonist is administered intramuscularly, intravenously, by intra-articular injection, subcutaneously, orally, or rectally.

The substance P antagonist is selected from the group including corticosteroids, Ondansetron, and 5-HT3-receptor antagonists. The Substance P antagonist is administered intramuscularly, intravenously, by intra-articular injection, subcutaneously, orally, or rectally.

The calcitonin gene-related peptide antagonist is administered intramuscularly, intravenously, by intra-articular injection, subcutaneously, orally, or rectally. The vasoactive intestinal peptide antagonist is administered intramuscularly, intravenously, by intra-articular injection, subcutaneously, orally, or rectally. The vasoactive intestinal peptide antagonist and is selected from the group including Botulinum toxin.

The vasoactive intestinal peptide antagonist is administered intramuscularly, intravenously, intrathecally, by intra-articular injection, subcutaneously, orally, or rectally.

The interleukin-4 antagonist is administered intramuscularly, intravenously, by intra-articular injection, subcutaneously, orally, or rectally.

The interleukin-6 antagonist and is selected from the group including bisphosphonates. The interleukin-6 antagonist is administered intramuscularly, intravenously, by intra-articular injection, subcutaneously, orally, or rectally. The interleukin-8 antagonist is administered intramuscularly, intravenously, by intra-articular injection, subcutaneously, orally, or rectally.

The kinin antagonist is administered intramuscularly, intravenously, by intra-articular injection, subcutaneously, orally, or rectally.

The serotonin receptor antagonist is administered intramuscularly, intravenously, intrathecally by intra-articular injection, subcutaneously, orally, or rectally.

The matrix metallo-proteinase antagonist is selected from the group including tetracyclines and macrolide antibiotics such as Clarithromycin. The matrix metallo-proteinase antagonist is administered intramuscularly, intravenously, intrathecally by intra-articular injection, subcutaneously, orally, or rectally.

The iNO, nitric oxide synthatse antagonist is administered intramuscularly, intravenously, intrathecally by intra-articular injection, subcutaneously, orally, or rectally.

Glial cell inflammatory transformational blockade involves cell receptor, gene transcriptional blockade and gene regulatory transformation blockade. The glial cell transformation antagonist is administered intramuscularly, intravenously, intrathecally by intra-articular injection, subcutaneously, orally, or rectally.

### DETAILED DESCRIPTION

The origins of pain are the biochemical mediators of inflammation. To treat pain, these mediators must be inhibited or blocked. The signals they send up through the nerve cells likewise must be blocked. The invention provides a method of treatment according to a model referred to as the Deagle Multilevel Inflammatory Molecular Pain Gate. This model maps pain transmission through a seven level hierarchy of pain gates. These seven levels are (1) the Langerhans complex in the grid patterns of the dermal-epidermal junction of the skin, (2) the dorsal root ganglion or DRG, (3) the substantia gelatinosa of the spinal cord, (4) ascending spinothalamic tracts, (5) subthalmic and thalamic nuclei, (6) the sensory receptive motor parietal-temporal cortical gate, and (7) the sensory-behavioral-motor frontal cortical gate. The model projects that a pain gate will only remain in the open state, allowing pain transmission with molecular mediators of inflammation, upon molecular actuation. The molecular activation of the pain process may be initiated at any level of the gating hierarchy, and spread to a contiguous or a non-contiguous region or mirror image. Thus pain can be a self-sustaining and propagating process, initiated and maintained as a separate and disease independent process, not dependent on tissue structural pathology, but sustained by a multilevel integrated hierarchy of neural gating processes dependent on inflammatory mediators.

The biochemical and physiologic changes (inflammatory mediators) that occur along the “pain pathway” (nociceptors, peripheral nerve, dorsal root ganglion, dorsal root, neurons in the spinal cord) may sensitize one or all these sites along the pain pathway and hence lead to chronic pain. Deagle's Law of the Multilevel Pain Gate states, “Changes at one level of the multilevel gate cause pathway and molecular facilitative neurotransmitter and inflammatory mediator changes at all other levels of the gated neural network from the Langerhans complex in the skin to the cortical levels.” Pain is a neurologic signal disease and may or may not be associated with tissue inflammation. Thus lack of structural disease or tissue inflammation in conditions such as fibromyalgia or other non-inflammatory pain disorders such as non-inflammatory neuropathy, are primarily due to molecular increased pain signal transmission. Irrespective of the type of pain, whether it is acute pain as in a sprain, sports injury or eurochange jellyfish sting, or whether it is chronic pain as in arthritis, migraine pain, back or neck pain from herniated disks, RSD/CRPS pain, migraine, fibromyalgia, interstitial cystitis, neuropathic pain, post-stroke pain etc, the underlying basis is the opening of the pain gate to allow the transmission of the pain signal from the primary or higher gates to the cortex at gate levels six and seven, where higher receptive, motor and behavioral responses occur.

A portion of the model known as Deagle's Law of the Multilevel Pain Gate states, “Any change in inflammatory or faciliatory gene and molecular mediators at one level of the neural multilevel pain gate, causes upregulation of the gate at all other levels from the Langerhans complexes in the skin to the highest cortical gating neural processes.” The present invention provides a method to blockade the pain gate at multiple levels, for acute and persistent chronic pain. Pharmaceuticals, immunopharmaceuticals, and gene control methodologies are provided to close the pain gate at any of the seven broad levels, which include the entire range of neural processing for the pain experience.

Thus, all pain is gated at the Langerhans complex in the skin, the dorsal root ganglion (DRG), the substantia gelatinosa, spinothalamic tracts, thalamic and other nuclei, receptive parietal-temporal cortex and motor-behavioral frontal cortex. Hence, each of these seven levels offers a specific opportunity to treat pain in a person in need of such treatment. For example, pain originating at a specific Langerhans complex can be treated at that specific, first level Langerhans complex in the skin. The successful treatment is applied only to this first level site, and the treatment may consist of a small dose of a suitable inhibitor, rather than a larger dose as might be required to inhibit pain by generalized treatment. The patient need not be broadly drugged, such as at the frontal cortex of the seventh level, to treat the first level source of pain.

The invention contemplates that acute or persistent pain disorder is best treated after evaluation of the origin. Treatment can be applied to any chosen level of the pain gate pathway. The chosen level may be selected as the lowest level where treatment appears practical. The chosen level may be selected according to the best available treatment, such a treatment directed to facilitator molecules of the chosen level. The treatment may employ one or more site-specific methodologies.

Molecules that close the gate can be considered primarily inflammatory or excitatory such as aspartate or glutamate, that operate via increasing nerve transmission, receptor density for NMDA and Na channels, and other excitatory neurotransmitters and peptides. This occurs by nerve and glial cell activation to upregulate and open the pain gate at the glial cell levels throughout the nervous system.

This model differs substantially from the model that inflammation is the sole cause of pain and that other molecular modulators of pain signal transmission or inhibition are not primary in the cause of chronic or acute pain disorders. This process involves pain gate facilitatory molecules. In other tissues and circumstances, these may be proinflammatory, or neutral or anti-inflammatory. Pain gate neuromodulatory signal facilatory and inhibitory as well as inflammatory molecular mediators of the gate include leukotrienes, prostaglandins, nitric oxide and the free radical NO, tumor necrosis factor alpha (TNF-Alpha), interleukin 1-alpha (IL1-Alpha), interleukin 1-beta (IL1-Beta), interleukin-4 (IL4), Interleukin-6 (IL-6) and interleukin-8 (IL-8), granulocyte inhibition factor (GIF), histamine and serotonin, substance P, matrix metalloproteinase, calcitonin gene-related peptide, vasoactive intestinal peptide (VIP), and the neuromodulatory inflammatory mediator peptide proteins neurokinin A, bradykinin, kallidin and T-kinin, and nitric oxide (NO) as well as the aspartate and glutamate via the N-Methyl D-Aspartate (NMDA) receptor or the AMPA or catecholamine receptors. These mediators alter pain transmission and inhibiton at one or more of the integrated vertical pain gates of the neural array. Thus pain is a signal and neural informational disorder resulting in increase pain nociceptive signal to the brain, and resulting in the brain and spinal cord sending increased vasconstrictive and muscular tonic spastic neurologic input to the blood vessels and muscles. Thus pain is efferent at seven levels to the cortex and afferent to the blood vessels and muscles as well as all levels of the gate.

Deagle's Law of the Multilevel Pain Gate states, “Changes at one level of the multilevel gate cause pathway and molecular neurotransmitter and inflammatory mediator changes at all other levels.” Pain can arise at any level of the gating process, with different molecules responsible, and different genetic, pharmaceutical, immunopharmaceutical and technical challenges to block the gate mediators. Pain of all types arises through the multilevel gate, with changes in molecular upregulation at all other continuous levels. Thus levels of molecules that open the gate in the skin are increased in fibromyalgia and skin levels of cytokines and excitatory neurotransmitters are increased in the brain and skin in spinal cord and peripheral nerve injuries.

Pain is a neurologic signal disease and may or may not be associated with tissue inflammation. Lack of structural disease or tissue inflammation in conditions such as fibromyalgia or other non-inflammatory pain disorders such as non-inflammatory neuropathy, are primarily due to molecular increased pain signal transmission. Irrespective of the type of pain, whether it is acute pain as in a sprain, sports injury or eurochange jellyfish sting, or whether it is chronic pain as in arthritis, migraine pain, back or neck pain from herniated disks, RSD/CRPS pain, migraine, fibromyalgia, interstitial cystitis, neuropathic pain, post-stroke pain, etc., the underlying basis is the opening of the pain gate to allow the transmission of the pain signal from the primary or higher gates to the cortex at gate levels six and seven, where higher receptive, motor and behavioral responses occur. This model differs substantially from the model that inflammation is the sole cause of pain and that other molecular modulators of pain signal transmission or inhibition are not primary in the cause of chronic or acute pain disorders.

In fibromyalgia, non-inflammatory neuromodulatory molecules are increased in the skin as well as in other disorders at the pain gates inflammatory molecules and profacilatory pain signal modulators are increased. Receptor density on the nerve fibers increases due to increased nerve traffic transmitting painful stimuli. Blockade of faciliative neurotransmitters and inflammatory gate controlling molecules narrows the twelve-lane superhighway facilitated for pain in the chronic pain state down to a two lane normal pathway, and reduces the receptor density for neurotransmitters as well.

This process involves pain gate facilitatory molecules. In other tissues and circumstances, these may be proinflammatory, or neutral, or anti-inflammatory. Irrespective of the characteristic of the pain, whether it is sharp, dull, aching, burning, stabbing, numbing or tingling, all pain arises with the allowance of increased signal of pain at one of the seven pain gate levels at the specific site of the structures that comprise the gates. In the skin this structure is the Langerhans dermal structures, next the dorsal root ganglion, the dorsal horn substantia gelatinosa, ascending spinothalamic tracts, subthalamic nuclei, sensory receptive cortex, and anterior motor and behavioral response cortex. Pain gate facilatatory molecules such as in the condition of fibromyalgia can be identified in the skin with increased frequency in this condition. Lack of pain gate inhibitory molecules may also be identified at all seven levels of the gate, with upregulation of genes, proteins, enzymes and glial cell transformation to open the gate in pain and down-regulation of genes, proteins, enzymes and blockade of glial cell transformation molecules in the pain state. Thus pain can be viewed as a signal disease and a neurological informational disorder, with excessive transmission to higher levels from one gate to another gate of pain signal increasingly processed, with lack of signal inhibition until there is a cortical response.

According to a study on neuropharmaceutical multilevel neural pain gate and transduction-transmission blockades, the neural process of pain requires multilevel hierarchical integrated gating and requires the pain gate to be opened by inflammatory molecules. These include interleukin 1 Beta, IL1B; necrosis factor alpha, TNF-Alpha; interleukin 6, IL6; interleukin 8, IL8; interleukin 2, IL2; and vanilloid receptor 1, VR1; and granulocyte inhibitor factor, GIF; iNO, glial cell activation, and free radical molecules such as hydroxyl OH, and nitric oxide or nitroperoxyl radicals NO, and NOOH.

These may be blocked with pharmaceuticals that bind to the receptor. Amgen has made Anakinra or Kineret to block IL1 Beta, and Etanercept and Enbrel to block TNF-Alpha.

Pain may be present in inflammatory or non-inflammatory conditions and therefore the presence of molecules that are found to cause or propagate pain if these molecules are not found at the pain gate. Non-inflammatory conditions such as fibromyalgia, myofascial pain, and non-inflammatory metabolic neuritic pain syndromes are excellent examples.

The invention proposes not only the use of PF, of preservative free forms for spinal and nerve blocks of these pharmaceuticals, but additional newer technologies, that would result in longer or permanent blockade of the pain pathways. The two-lane bicycle pathway of pain has been widened to a twelve lane concrete superhighway, and must be made to return to the normal appropriate physiology of the two lane bicycle pathway.

These new pharmaceutical and immunopharmaceutical technologies and uses include:

1. Monoclonal antibodies to the receptors would bind and make the receptor temporarily or permanently inactivated and unable to keep the pain gate open.

2. Soluble receptor blockade would bind free cytokines in paragraph one, and prevent interaction with the receptors.

3. Antibodies to the cytokines would bind them on site—this is much more temporary. The best and most permanent neuropharmaceutical would be #1.

4. Antisense RNA would block gene transcription for the making of more cytokines—This could be injected at the specific nerve cord peripheral levels, and turns off the ability of the body to make excessive local cytokines that keep the pain gate open.

5. Glial cell activation blockade—A large number of current drugs blocks the supporting and nourishing glial cells around the nerves of the body, spinal cord and brain. These cells are the primary generators of inflammatory cytokine molecules that keep the pain gate open. Changes in the cellular metabolism are known to be blocked by many older drugs, and newer more permanent blocking drugs or immunopharmaceuticals, antisense-RNA blockade, etc. will render glial cells incapable of transformation or reverse these changes back to the non-open state. Drugs currently known to perform this action with published electron microscopic and immunologic verification include Propentofylline, Minocycline, Tetracycline, Doxycycline, and Lefluonomide and its active metabolite A77 1726.

6. Lipoxygenase and cycloxygenase blockade with local NSAIDS, non steroidal antiinflammatory molecules, antisense RNA blockade, and monoclonal antibodies to the glial cell enzyme receptors that regulate these enzymes.

7. Inducible NO, nitrous oxide synthase—The production of NO, or nitrous oxide, is pro-inflammatory. Blocking excessive induction with iNO blocking drugs known now, and future enzyme blockade with monoclonal antibodies or antisense RNA blockade or other means of gene transcription blockade in the endoplasmic reticulum, will down-regulate over activity demonstrated with increased pain, inflammation and eventual nerve cell death or apoptosis.

8. Anti-inflammatory cytokine—Anti-inflammatory cytokine analogues made with recombinant DNA could produce pharmaceutical molecular equivalents of Interleukin 10 (IL10), the most powerful anti-inflammatory cytokine, with properties that would last many times longer than the natural molecule. This would act synergist with other blockade types. IL2 has also been demonstrated to have antinoceptive properties. Gene insertion and manipulation to increase these anti-inflammatory factors are prime targets for interventional therapies with directed neuropharmaceuticals based on gene control, which would be very effective in reduction of pain. These pain gate inhibitory molecules close the molecular transmission of pain signal. Thus pain can be viewed as a signal disease and an neurological informational disorder, with excessive transmission to higher levels from one to another gate of pain signal increasing processed, with lack of signal inhibition until there is a cortical response.

Free Radical blockade—DMSO is know to topically stop CRP1 pain or RSD, pain syndromes by blocking free hydroxyl (OH), and thus to stop pain. It also works in fibromyalgia, where in about 30 percent of these patients' elevations in skin cytokine levels can be demonstrated. Thus I have developed a topical with DMSO, 2% DL-Phenylalanine to block the opoid receptor, 2% Anakinra to block Interleukin1, 2% Trental or pentoxyphylline to block TNF-Alpha. The invention includes other free radical scavengersds and newer gene regulators such as antisense RNA and other pharmaceuticals and immunopharmaceuticals that block specific genes that allow free radical generation.

The acid-sensing ion channel family (ASIC) comprises six discrete ASIC subunits (ASIC1A, ASIC1B, ASIC2A, ASIC2B, ASIC3, ASIC4) that detect tissue acidosis (i.e. a decrease in pH), a hallmark response to tissue injury, pain and inflammation. Like many ion channels, ASICs are multimeric protein complexes, with four or more ASIC proteins physically associating to form the functional ion channel. The individual ASIC subunits can be all identical (“homomers”) or different (“heteromers”). A correlation between pain intensity/discomfort and the degree of local acidification is well established as exemplified by the intense pain associated with muscle cramps, resulting from lactic acid accumulation in muscle tissue. Tissue acidosis also occurs in many chronic pain conditions including angina, stroke, ischemic heart disease, arthritis, cancer, infections, and traumatic injuries.

All six ASIC receptor subtypes are located within sensory neurons, with ASIC1B and AISC3 showing the highest degree of selectivity for sensory neurons. When expressed in vitro in cultured cell lines, ASICs can be activated by acid solutions (low pH) and generate depolarizing currents similar to the native currents recorded from intact sensory neurons. ASIC1 channels activate and inactivate very rapidly even when the medium remains acidic. In contrast, ASIC3 channels display biphasic currents with a sustained phase during which channels remain open as long as the pH is low, making this receptor a key mediator of pain during sustained acidosis. The ASIC3 channel has also been linked to ischemic heart pain and inflammatory bowel disease. Data from ASIC3 knockout mice have also confirmed that the ASIC3 channel is an important component of the acid-sensing pain response.

Amiloride is the only known compound that blocks ASICs. However, the effect is not potent and not selective as indicated by amiloride's potent blockade of other receptor targets, which precludes its clinical use as an ASIC antagonist.

Neurotrophins are a family of structurally similar proteins that regulate the growth, differentiation, function, survival and plasticity of nerve cells. These proteins are expressed in greatest abundance within the nervous system, including target tissues for sensory nerve endings. Neurotrophins produce their effects in responsive cells by interacting with two classes of cell surface receptors: the selective trk receptors (trkA, trkB and trkC) and the non-selective p75^{NGF} receptors, which do not discriminate between the various neurotrophin proteins. Nerve Growth Factor (NGF) is the prototypic member of the neurotrophin family, having been discovered over 40 years ago. The dysfunction of NGF-mediated signaling has been implicated in disorders such as chronic pain, ALS, Parkinson's disease and stroke.

There is now extensive evidence that neurotrophins alter the functions of nerve cells that recognize painful stimuli (nociceptors). Specifically, NGF binding to trkA/p75^{NGF} has been shown to have both acute and long-term effects on nociceptor function. Tissue damage or inflammation induces high levels of NGF secretion. The acute effect of NGF is to stimulate the release of naturally occurring chemicals that increase the sensitivity of nociceptors to pain (e.g. substance P, CGRP, histamine). After this initial phase, the over-stimulation of NGF receptors leads to a remodeling of pain pathways with an increase in the number of nociceptive fibers and pain receptors, such as ion channels. These acute and long term changes in the processing of pain signals and reorganization of the neuronal networks mediated by NGF underlie the chronic pain mechanisms induced by nerve damage or disease.

Another study has found that a polypharmaceutical approach is superior and a multilevel blockade is superior. Polypharmaceutical approaches are additive and multiplicative in action. Thus current studies with IL1 and TNF-Alpha blockade in the cerebrospinal fluid are additive and thus would result in much more efficacious and profound blockades that last many times longer. Blockades with multiple pharmaceuticals and at multiple levels are much more effective than with single agents and at single levels. This invention contemplates the use of current immunopharmaceuticals that block cytokines, pharmaceuticals that block numerous receptors that allow pain transmission, and future technologies for anti-sense RNA, gene insertion to block pain gate faciliatory molecules or gene manipulation to increase pain gate inhibitory molecules.

The origins of pain are the biochemical mediators pain signal facilitation and may also include some mediator molecules that have the property of inflammation and the inflammatory response. Modulators thus may not have any affect directly or indirectly on inflammation in non-inflammatory pain syndromes, or in part of the processes that are facilitated that are non-inflammatory in situations where some of the molecular factors perpetuating the pain are inflammatory. This model is thus more inclusive of all modulatory molecules, both non-inflammatory and inflammatory. Nociceptors in the skin, ligaments, periosteum, nerve sheath and capsules of internal organs are some of the sources of pain signals. The pain gate is opened by the presence of inflammatory molecules. Closing the pain gate stops the transmission from local structures initiating the pain signal.

The new Multilevel Cytokine Pain Gate Theory of Deagle proposes that the first gate is the Langerhans structures in a grid-like pattern at the dermal-epidermal junction. Autonomic, sensory and microvascular and local cytokine microorganelle structures are the first step at which inflammatory mediators such as LOX and COX leukotrienes, protaglandins, and cytokines, NO nitric oxide, free radical inflammatory molecules as well as neurotransmitters, especially NMDA N-Methyl D-Aspartate, and others, locally modulate the pain signal.

The next pain gate integrated is the dorsal root ganglion (DRG). The spinothalamic tracks and interneurons at the substantia gelatinosa. Spinal recruitement of pain at this level connects to gates at levels above and below the levels at which pain signal enters. At each level, the pain can be primarily perpetuated, meaning that a pain signal does not require a peripheral stimulus to perpetuate, explaining the presence of pain without significant pathology, such as normal MRI or x-ray study.

Inflammation occurs when there is infection or tissue injury. Tissue injury may arise from a physical, chemical or biological trauma or irritation. Degeneration of tissue subsequent to aging or previous injury can also lead to inflammation. Injured tissues can be muscle, ligament, disks, joints or nerves. A variety of mediators are generated by tissue injury and inflammation. These include substances produced by damaged tissue, substances of vascular origin as well as substances released by nerve fibers themselves, sympathetic fibers and various immune cells. There are three phases of an inflammatory response: initiation, maintenance and termination. Upon tissue injury or painful stimulation, specialized blood cells in the area such as basophils, mast cells and platelets release inflammatory mediators serotonin, histamine and nitric oxide. Subsequent to the binding of serotonin to its receptor, there is inflammation of the adjacent nerves and the nerve endings release short-lived inflammatory peptide proteins such as substance P and Calcitonin gene-related peptide (CGRP). In addition, clotting factors in the blood produce and activate potent inflammatory mediator peptide proteins called neurokinin A, bradykinin, kallidin and T-kinin. All of these proteins increase blood flow to the area of injury, stimulate arachidonic acid metabolism to generate inflammatory mediators prostaglandins and attract specialized immune cells to the area. The first immune cells to the area are tissue macrophages, which provide the front line defense against bacterial infection. Macrophages release powerful enzymes to digest any bacteria that are present and produce potent inflammatory chemical mediators (called cytokines) to attract and activate other cells of the immune system.

Shortly thereafter the area of bacterial invasion or tissue injury is invaded by the other immune cells, which include white blood cells such as T helper cells, lymphocytes, neutrophils, eosinophils, and other cells such as fibroblasts and endothelial cells. These immune cells respond to the chemical mediators, release destructive enzymes to kill any invading organism and release more chemical mediators to attract more immune cells. A consequence of this immune response is tissue damage, pain and spasm. In a sense the initial immune reaction ignites a cascade of immune reactions and generates an inflammatory soup of chemical mediators. These chemical mediators produced by the immune cells include prostaglandin, nitric oxide, tumor necrosis factor alpha, interleukin 1-alpha, interleukin 1-beta, interleukin-4, interleukin-6 and interleukin-8, histamine, and serotonin. In the area of injury and subsequently in the spinal cord, enzymes such as cyclooxygenase increase the production of these inflammatory mediators. These chemical mediators attract tissue macrophages and white blood cells to localize in an area to engulf (phagocytize) and destroy foreign substances.

The chemical mediators released during the inflammatory response give rise to the typical findings associated with inflammation. The inflammatory mediators produce several effects. Excitation of these pain nociceptive receptors stimulate the specialized nerves, e.g., C fibers and A-delta fibers that carry pain impulses to the spinal cord and brain. Subsequent to tissue injury, the expression of sodium channels in nerve fibers is altered significantly thus leading to abnormal excitability in the sensory neurons. Nerve impulses arriving in the spinal cord stimulate the release of inflammatory protein substance P. The presence of substance P and other inflammatory proteins such as calcitonin gene-related peptide (CGRP), neurokinin A, and vasoactive intestinal peptide removes magnesium induced inhibition and enables excitatory inflammatory proteins such as glutamate and aspartate to activate specialized spinal cord NMDA receptors. This results in magnification of all nerve traffic and pain stimuli that arrive in the spinal cord from the periphery.

Activation of motor nerves that travel from the spinal cord to the muscles results in excessive muscle tension. More inflammatory mediators are released which then excite additional pain receptors in muscles, tendons and joints, generating more nerve traffic and increased muscle spasm. Persistent abnormal spinal reflex transmission due to local injury or even inappropriate postural habits may then result in a vicious circle between muscle hypertension and pain.

Separately, constant C-fiber nerve stimulation to transmission pathways in the spinal cord result in even more release of inflammatory mediators, but this time within the spinal cord. Inflammation causes increased production of the enzyme cyclooxygenase-2 (Cox-2), leading to the release of chemical mediators both in the area of injury and in the spinal cord. Widespread induction of Cox-2 expression in spinal cord neurons and in other regions of the central nervous system elevates inflammatory mediator prostaglandin E. (PGE) levels in the cerebrospinal fluid.

The major inducer of central Cox-2 upregulation is inflammatory mediator interleukin-1 beta in the CNS. Basal levels of the enzyme phospholipase A activity in the CNS do not change with peripheral inflammation.

Abnormal development of sensory-sympathetic connections follow nerve injury, and contribute to the hyperalgesia (abnormally severe pain) and allodynia (pain due to normally innocuous stimuli). These abnormal connections between sympathetic and sensory neurons arise in part due to sprouting of sympathetic axons. Studies have shown that sympathetic axons invade spinal cord dorsal root ganglia (DRG) following nerve injury, and activity in the resulting pericellular axonal “baskets” may underlie painful sympathetic-sensory coupling. Sympathetic sprouting into the DRG may be stimulated by neurotrophins such as nerve growth factor (NGF), brain derived neurotrophic factor (BDNF), neurotrophin-3 (NT-3) and neurotrophin 4/5 (NT-4/5).

The central nervous system response to pain can keep increasing even though the painful stimulus from the injured tissue remains steady. This “wind-up” phenomenon in deep dorsal neurons can dramatically increase the injured person's sensitivity to the pain. Local tissue inflammation can also result in pain hypersensitivity in neighboring uninjured tissue (secondary hyperalgesia) by spread and diffusion of the excess inflammatory mediators that have been produced as well as by an increase in nerve excitability in the spinal cord (central sensitization). This can result in a syndrome comprising diffuse muscle pain and spasm, joint pain, fever, lethargy and anorexia.

The inflammatory mediators interact in a complex way to induce, enhance and propagate persistent pain. There are also natural anti-inflammatory mediators produced by the body to cool down inflammation and the inflammatory response. Interleukin-1 beta is a potent pain-generating mediator. Two pain producing pathways have been identified: Inflammatory stimuli or injury to soft tissue induces the production of mediator Bradykinin, which stimulates the release of mediator Tumor necrosis factor alpha. The TNF-alpha induces production of (i) Interleukin-6 and Interleukin-1-Beta which stimulate the production of cyclooxygenase enzyme products, and (ii) Inflammatory mediator Interleukin-8, which stimulates production of sympathomimetics (sympathetic hyperalgesia). Effects of Interleukin-1 beta include: Interleukin-1 beta stimulates inflammatory mediators prostaglandin E (PGE), cyclooxygenase-2 (COX-2) and matrix metalloproteases (MMPs) production. Interleukin-1 beta is a significant catalyst in cartilage damage. It induces the loss of proteoglycans, prevents the formation of the cartilage matrix, and prevents the proper maintenance of cartilage.

Interleukin-1 beta is a significant catalyst in bone resorption. It stimulates osteoclasts cells involved in the resorption and removal of bone. Interleukin-6 is another potent pain-generating inflammatory mediator. A significant amount of Interleukin-6 is produced in the rat spinal cord following peripheral nerve injury that results in pain behaviors suggestive of neuropathic pain. These spinal IL-6 levels correlated directly with the mechanical allodynia intensity following nerve injury.

Interleukin-8 is a pain-generating inflammatory mediator. In one study of patients with post herpetic neuralgia, the patients who received methylprednisolone, had interleukin-8 concentrations decrease by 50 percent, and this decrease correlated with the duration of neuralgia and with the extent of global pain relief. (p<0.001 for both comparisons).

Interleukin-10 is one of the natural anti-inflammatory cytokines, which also include Interleuken-1 receptor antagonist (IL-1ra), Interleukin-4, Interleukin-13 and transforming growth factor-betaI (TGF-betaI). Interleukin-10 (IL-10) is made by immune cells called macrophages during the shut-off stage of the immune response. Interleukin-10 is a potent anti-inflammatory agent, which acts partly by decreasing the production of inflammatory cytokines interleukin-1 beta (Interleukin-1 beta), tumor necrosis factor-alpha (TNF-alpha) and inducible nitric oxide synthetase (iNOS), by injured nerves and activated white blood cells, thus decreasing the amount of spinal cord and peripheral nerve damage.

In rats with spinal cord injury (SCI), a single injection of IL-10 within half an hour resulted in 49% less spinal cord tissue loss than in untreated rats. Rresearchers observed nerve fibers traveling straight through the spared tissue regions, across the zone of injury. They also reported a decrease in the inflammatory mediator TNF-alpha, which rises significantly after SCI.

Prostaglandins are inflammatory mediators that are released during allergic and inflammatory processes. Phospholipase A2 enzyme, which is present in cell membranes, is stimulated or activated by tissue injury or microbial products. Activation of phospholipase A2 causes the release of arachidonic acid from the cell membrane phospholipid. From here there are two reaction pathways that are catalyzed by the enzymes cyclooxygenase (COX) and lipoxygenase (LOX). These two enzyme pathways compete with one another. The cyclooxygenase enzyme pathway results in the formation of inflammatory mediator prostaglandins and thromboxane. The lipoxygenase enzyme pathway results in the formation of inflammatory mediator leukotriene. Because they are lipid soluble, these mediators can easily pass out through cell membranes. In the cyclooxygenase pathway, the prostaglandins D, E and F plus thromboxane and prostacyclin are made.

Thromboxanes are made in platelets and cause constriction of vascular smooth muscle and platelet aggregation. Prostacyclins, produced by blood vessel walls, are antagonistic to thromboxanes as they inhibit platelet aggregation. Prostaglandins have diverse actions dependent on cell type but are known to generally cause smooth muscle contraction. They are very potent but are inactivated rapidly in the systemic circulation.

Leukotrienes are made in leukocytes and macrophages via the lipoxygenase pathway. They are potent constrictors of the bronchial airways. They are also important in inflammation and hypersensitivity reactions as they increase vascular permeability and attract leukocytes.

Tumor necrosis factor alpha is an inflammatory mediator that is released by macrophages as well as nerve cells. Very importantly, TNF-alpha is released from injured or herniated disks.

During an inflammatory response, nerve cells communicate with each other by releasing neuro-transmitter glutamate. This process follows activation of a nerve cell receptor called CXCR4 by the inflammatory mediator stromal cell-derived factor 1 (SDF-1). An extraordinary feature of the nerve cell communication is the rapid release of inflammatory mediator tumor necrosis factor-alpha (TNF alpha). Subsequent to release of TNF-alpha, there is an increase in the formation of inflammatory mediator prostaglandin. Excessive prostaglandin release results in an increased production of neurotransmitter glutamate and an increase in nerve cell communication resulting in a vicious cycle of inflammation. There is excitation of pain receptors and stimulation of the specialized nerves, e.g., C fibers and A-delta fibers that carry pain impulses to the spinal cord and brain. Studies have established that herniated disk tissue (nucleus pulposus) produces a profound inflammatory reaction with release of inflammatory chemical mediators. Disk tissue applied to nerves may induce a characteristic nerve sheath injury, increased blood vessel permeability, and blood coagulation. The primary inflammatory mediator implicated in this nerve injury is tumor necrosis factor-alpha, but other mediators including interleukin 1-beta may also participate in the inflammatory reaction.

Recent studies have also shown that that local application of nucleus pulposus may induce pain-related behavior in rats, particularly hypersensitivity to heat and other features of a neuropathic pain syndrome.

Nitric oxide is an inflammatory mediator that is released by macrophages. Other mediators of inflammation such as reactive oxygen products and cytokines considerably contribute to inflammation and inflammatory pain by causing an increased local production of cyclooxygenase enzyme. The cyclooxygenase enzyme pathway results in the formation of inflammatory mediator prostaglandins and thromboxane. Concurrently to the increased production of the cyclooxygenase-2 (COX-2) gene, there is increased production of the gene for the enzyme inducible nitric oxide synthetase (iNOS), leading to increased levels of nitric oxide (NO) in inflamed tissues. In these tissues, NO has been shown to contribute to swelling, hyperalgesia (heightened reaction to pain) and pain. NO localized in high amounts in inflamed tissues has been shown to induce pain locally and enhances central as well as peripheral stimuli. Inflammatory NO is thought to be synthesized by the inducible isoform of nitric oxide synthetase (iNOS).

Substance P (sP) is an important early event in the induction of neuropathic pain states. It is the release of Substance P from injured nerves which then increases local tumor necrosis factor alpha (TNF-alpha) production. Substance P and TNF-alpha then attract and activate immune monocytes and macrophages, and can activate macrophages directly. Substance P effects are selective and Substance P does not stimulate production of interleukin-1, interleukin-3, or interleukin-6. Substance P and the associated increased production of TNF-alpha has been shown to be critically involved in the pathogenesis of neuropathic pain states. TNF protein and message are then further increased by activated immune macrophages recruited to the injury site several days after the primary injury. TNF-alpha can evoke spontaneous electrical activity in sensory C and A-delta nerve fibers that results in low-grade pain signal input contributing to central sensitization. Inhibition of macrophage recruitment to the nerve injury site, or pharmacologic interference with TNF-alpha production has been shown to reduce both the neuropathologic and behavioral manifestations of neuropathic pain states.

Gelatinase B or matrix metallo-proteinase 9 (MMP-9) is an enzyme that is one of a group of metalloproteinases (which includes collagenase and stromelysin) that are involved in connective tissue breakdown. Normal cells produce MMP-9 in an inactive or latent form. The enzyme is activated by inflammatory mediators such as TNF-alpha and interleukin-1 that are released by cells of the immune system (mainly neutrophils but also macrophages and lymphocytes) and transformed cells. MMP-9 helps these cells migrate through the blood vessels to inflammatory sites or to metastatic sites. Activated, MMP-9 can also degrade collagen in the extra cellular matrix of articular bone and cartilage and is associated with joint inflammation and bony erosions. Consequently, MMP-9 plays a major role in acute and chronic inflammation, in cardiovascular and skin pathologies as well as in cancer metastasis. MMP-9 can also degrade a protein called beta amyloid. Normal cells produce MMP-9 in an inactive, or latent form, converting it to active enzyme when it is needed. But when normal brain cells producing MMP-9 fail to activate the enzyme, insoluble amyloid-b could accumulate in brain tissue. Previous research has shown that the undegraded form of amyloid-beta accumulates in the brain as senile “plaques” that signal the presence of Alzheimer's disease.

The inflammatory response ends by immune cells producing anti-inflammatory cytokine mediators that help to suppress the inflammatory response and suppress the production of pro-inflammatory cytokines. The natural anti-inflammatory cytokines are interleuken-1 receptor antagonist (IL-1ra), interleukin-10, interleukin-4, interleukin-13 and transforming growth factor-betaI (TGF-betaI). Research has shown that administration of these anti-inflammatory cytokines prevents the development of painful nerve pain that is produced by a naturally occurring irritant protein called dynorphin A. Under normal circumstances, the inflammatory response should only last for as long as the infection or the tissue injury exists. Once the threat of infection has passed or the injury has healed, the area should return to normal existence. One of the ways that the inflammatory response ends is by a phenomenon known as “apoptosis.” Most of the time, cells of the body die by being irreparably damaged or by being deprived of nutrients. This is known as necrotic death. However, cells can also be killed in another way, i.e., by “committing suicide.” On receipt of a certain chemical signal, most cells of the body can destroy themselves. This is known as apoptotic death.

There are two main ways in which cells can commit apoptosis. The first is by receiving an apoptosis signal. When a chemical signal is received that indicates that the cell should kill itself, it does so. The second is by not receiving a “stay-alive” signal. Certain cells, once they reach an activated state, are primed to kill themselves automatically within a certain period of time, i.e., to commit apoptosis, unless instructed otherwise. However, there may be other cells that supply them with a “stay-alive” signal, which delays the apoptosis of the cell. It is only when the primed cell stops receiving this “stay-alive” signal that it kills itself.

The immune system employs method two above. The immune cells involved in the inflammatory response, once they become activated, are primed to commit apoptosis. Helper T cells emit the stay-alive signal, and keep emitting the signal for as long as they recognize foreign antigens or a state of injury in the body, thus prolonging the inflammatory response. It is only when the infection or injury has been eradicated, and there is no more foreign antigen that the helper T cells stop emitting the stay-alive signal, thus allowing the cells involved in the inflammatory response to die off. If foreign antigen is not eradicated from the body or the injury has not healed, or the helper T cells do not recognize that fact, or if the immune cells receive the stay-alive signal from another source, then chronic inflammation may develop.

The final pathway for the natural suppression of the inflammatory response is in the spinal cord where there is a complex network of inhibitory neurons (gate control) that is driven by descending projections from brain stem sites. These inhibitory neurons act to dampen and counteract the spinal cord hyper excitability produced by tissue or nerve injury. Thus, peripherally evoked pain impulses pass through a filtering process involving inhibitory transmitters gamma-aminobutyric acid (GABA), glycine and enkephalins. The activity of these substances in the spinal cord usually attenuates and limits the duration of pain. In the case of persistent pain, there is evidence of pathological reduction of the supraspinal inhibitory actions in combination with ectopic afferent input in damaged nerves.

Arthritis is an inflammatory pain syndrome involving inflammation of the joints. People of all ages including children and young adults can develop arthritis. The symptoms are intermittent pain, swelling, redness and stiffness in the joints. There are many different types of arthritis, some of which are rheumatoid arthritis, osteoarthritis, infectious arthritis and spondylitis. In rheumatoid arthritis, and other autoimmune diseases like systemic lupus erythematosus (SLE), the joints are destroyed by the immune system. In osteoarthritis, the biggest risk factor is a previous injury to the joint, ligament or cartilage. Injuries that seem to heal perfectly well appear to set up a process of deterioration that can produce severe pain and disability decades later. The injury need not be sustained in one episode. Long term or repeated trauma can have the same effect.

TNF-alpha and interleukin 1-beta play an important role in rheumatoid arthritis by mediating cytokines that cause inflammation and joint destruction. TNF-alpha, interleukin 1-beta and substance P are elevated in the joint fluids in patients with rheumatoid arthritis. These inflammatory mediators are also elevated in the joint fluid in patients with osteoarthritis albeit to a far less extent. Along with mechanical factors, growth factors and cytokines such as TGF beta 1, IL-1 alpha, IL-1 beta and TNF-alpha may be involved in the formation and growth of osteophytes, since these molecules can induce growth and differentiation of mesenchymal cells. The incidence and size of osteophytes may be decreased by inhibition of direct or indirect effects of these cytokines and growth factors on osteoid deposition in treated animals. Inhibition of interleukin-1 receptor also decreases the production of metalloproteinase enzymes collagenase-1 and stomelysin-1 in the synovial membrane and cartilage. These enzymes are involved in connective tissue breakdown.

Back and neck pain most commonly results from injury to the muscle, disk, nerve, ligament or facet joints with subsequent inflammation and spasm. Degeneration of the disks or joints produces the same symptoms and occurs subsequent to aging, previous injury or excessive mechanical stresses that this region is subjected to because of its proximity to the sacrum in the lower back. Herniated disk tissue (nucleus pulposus) produces a profound inflammatory reaction with release of inflammatory chemical mediators most especially tumor necrosis factor alpha. Subsequent to release of TNF-alpha, there is an increase in the formation of inflammatory mediator prostaglandin and nitric oxide.

It is now known that tumor necrosis factor alpha is released by herniated disk tissue (nucleus pulposus), and is primarily responsible for the nerve injury and behavioral manifestations of experimental sciatica associated with herniated lumbar discs. This has been confirmed by numerous animal studies and research wherein application of disk tissue (nucleus pulposus) to a nerve results in nerve fiber injury, with reduction of nerve conduction velocity, intracapillary thrombus formation, and the intraneural edema formation. One study demonstrated that disk tissue (nucleus pulposus) increases inducible nitric oxide synthetase activity in spinal nerve roots and that nitric oxide synthetase inhibition reduces nucleus pulposus-induced swelling and prevents reduction of nerve-conduction velocity. According to the authors, the results suggest that nitric oxide is involved in the pathophysiological effects of disk tissue (nucleus pulposus) in disc herniation.

Tumor necrosis factor alpha and other inflammatory mediators induce phospholipase A2 activation. High levels of phospholipase A2 previously have been demonstrated in a small number of patients undergoing lumbar disc surgery. Phospholipase A2 is the enzyme responsible for the liberation of arachidonic acid from cell membranes at the site of inflammation and is considered to be the limiting agent in the production of inflammatory mediator prostaglandins and leukotrienes.

Subsequent to the release of inflammatory mediators, activation of motor nerves that travel from the spinal cord to the muscles results in excessive muscle tension, spasm and pain. The vast majority of herniated disk pain is inflammatory in origin, can be treated medically and does not require surgery. Surgery is only indicated when there is compression of the nerve roots producing significant muscle weakness or urinary or bowel incontinence.

Fibromyalgia is a chronic, painful musculoskeletal disorder characterized by widespread pain, pressure hyperalgesia, morning stiffness, sleep disturbances including restless leg syndrome, mood disturbances, and fatigue. Other syndromes commonly associated with fibromyalgia include irritable bowel syndrome, interstitial cystitis, migraine headaches, temporomandibular joint dysfunction, dysequilibrium including nerve mediated hypotension, sicca syndrome, and growth hormone deficiency. Fibromyalgia is accompanied by activation of the inflammatory response system, without immune activation. In fact, there is some evidence that fibromyalgia is accompanied by some signs of immunosuppression. Several studies have shown that there are increased levels of the inflammatory transmitter substance P (SP) and calcitonin gene related peptide (CGRP) in the spinal fluid of patients with fibromyalgia syndrome (FMS). The levels of platelet serotonin are also abnormal. Furthermore, in patients with fibromyalgia, the level of pain intensity is related to the spinal fluid level of arginine, which is a precursor to the inflammatory mediator nitric oxide (NO). Another study found increases over time in blood levels of cytokines interleukin-6, interleukin-8 and interleukin-1R antibody (IL-1Ra) whose release is stimulated by substance P. The study authors concluded that because interleukin-8 promotes sympathetic pain and interleukin-6 induces hypersensitivity to pain, fatigue and depression, both cytokines play a role in producing FM symptoms.

The painful neurogenic vasodilation of meningeal blood vessels is a key component of the inflammatory process during migraine headache. The cerebral circulation is supplied with two vasodilator systems: the parasympathetic system storing vasoactive intestinal peptide, peptide histidine isoleucine, acetylcholine and in a subpopulation of nerves neuropeptide Y, and the sensory system, mainly originating in the trigeminal ganglion, storing inflammatory mediator substance P, neurokinin A and calcitonin gene-related peptide (CGRP). A clear association between migraine and the release of inflammatory mediator calcitonin gene-related peptide (CGRP) and substance P (SP) has been demonstrated. Jugular plasma levels of the potent vasodilator, calcitonin gene-related peptide (CGRP) have been shown to be elevated in migraine headache. CGRP-mediated neurogenic dural vasodilation is blocked by anti-migraine drug dihydroergotamine, triptans, and opioids. In cluster headache and in chronic paroxysmal hemicrania, there is additional release of inflammatory mediator vasoactive intestinal peptide (VIP) in association with facial symptoms (nasal congestion, runny nose).

Immunocytochemical studies have revealed that cerebral blood vessels are invested with nerve fibers containing inflammatory mediator neuropeptide Y (NPY), vasoactive intestinal peptide (VIP), peptide histidine isoleucine (PHI), substance P (SP), neurokinin A (NKA), and calcitonin gene-related peptide (CGRP). In addition, there are studies reporting the occurrence of putative neurotransmitters such as cholecystokinin, dynorphin B, galanin, gastrin releasing peptide, vasopressin, neurotensin, and somatostatin. The nerves occur as a longitudinally oriented network around large cerebral arteries. There is often a richer supply of nerve fibers around arteries than veins. The origin of these nerve fibers has been studied by retrograde tracing and denervation experiments. These techniques, in combination with immunocytochemistry, have revealed a rather extensive innervation pattern. Several ganglia, such as the superior cervical ganglion, the sphenopalatine ganglion, the otic ganglion, and small local ganglia at the base of the skull, contribute to the innervation. Sensory fibers seem to derive from the trigeminal ganglion, the jugular-nodose ganglionic complex, and from dorsal root ganglia at the cervical spine level C2. The noradrenergic and most of the NPY fibers derive from the superior cervical ganglion. A minor population of the NPY-containing fibers contains vasoactive intestinal peptide (VIP), instead of NA and emanates from the sphenopalatine ganglion. The cholinergic and the vasoactive intestinal peptide (VIP)-containing fibers derive from the sphenopalatine ganglion, the otic ganglion, and from small local ganglia at the base of the skull. Most of the substance P (SP-), neurokinin A (NKA), and calcitonin gene-related peptide (CGRP)-containing fibers derive from the trigeminal ganglion. Minor contributions may emanate from the jugular-nodose ganglionic complex and from the spinal dorsal root ganglia. Neuropeptide Y (NPY), is a potent vasoconstrictor in vitro and in situ. Vasoactive intestinal peptide (VIP), peptide histidine isoleucine (PHI), substance P (SP), neurokinin A (NKA), and calcitonin gene-related peptide (CGRP) act via different mechanisms to induce cerebrovascular dilatation.

Meningeal blood vessels are involved in the generation of migraine pain and other headaches. Classical experiments have shown that blood vessels of the cranial dura mater are the most pain-sensitive intracranial structures. Dural blood vessels are supplied by trigeminal nerve fibers, and dilate in response to activation of the trigeminal nerves and release of neuropeptide cytokines such as substance P (SP) and calcitonin gene-related peptide (CGRP). CGRP can be released experimentally from dural nerve fibers, and there is evidence that this occurs also during migraine attacks. Stimulation of dural nerve fibers causes vasodilatation and an increase in dural arterial flow, which depends on the release of CGRP but not SP. On the other hand, SP is known to mediate plasma leakage (extravasation) from small veins in the dura mater. The dural arterial flow depends also on the formation of cell wall nitric oxide. The introduction of serotonin (5-HT.sub.1) receptor agonists such as sumatriptan changed the treatment strategies for migraine. Sumatriptan and other triptans may inhibit the release of inflammatory mediators from the trigeminal nerve. Sumatriptan has been shown to block the release of vasoactive cytokines from trigeminal nerves that surround the blood vessels in the dura mater during migraine. Sumatriptan blocks nerve fiber induced plasma extravasation but has only minor effects on nerve fiber mediated vasodilatation and dural arterial flow.

Foods like cheese, beer, and wine can also induce migraine in some people because they contain the mediator histamine and/or mediator-like compounds that cause blood vessels to expand. Women tend to react to histamine-containing foods more frequently than men do, on account of a deficiency in an enzyme (diamine oxidase) that breaks histamine down. Taking supplemental B6 has been shown to be helpful in migraine, as it can increase diamine oxidase activity.

Nerve (Neuropathic) pain syndromes include carpal tunnel syndrome, trigeminal neuralgia, post herpetic neuralgia, and phantom limb pain. Nociceptive pain is mediated by receptors on A-delta and C nerve fibers, which are located in skin, bone, connective tissue, muscle and viscera. These receptors serve a biologically useful role at localizing noxious chemical, thermal and mechanical stimuli. Nociceptive pain can be somatic or visceral in nature. Somatic pain tends to be well-localized, constant pain that is described as sharp, aching, throbbing, or gnawing. Visceral pain, on the other hand, tends to be vague in distribution, spasmodic in nature and is usually described as deep, aching, squeezing and colicky in nature. Examples of nociceptive pain include: post-operative pain, pain associated with trauma, and the chronic pain of arthritis. Neuropathic pain, in contrast to nociceptive pain, is described as “burning”, “electric”, “tingling”, and “shooting” in nature. It can be continuous or paroxysmal in presentation. Whereas nociceptive pain is caused by the stimulation of peripheral A-delta and C-polymodal pain receptors, by inflammatory mediators, (e.g., histamine bradykinin, substance P, etc.) neuropathic pain is produced by injury or damage to peripheral nerves or the central nervous system. The hallmarks of neuropathic pain are chronic allodynia and hyperalgesia. Allodynia is defined as pain resulting from a stimulus that ordinarily does not elicit a painful response (e.g. light touch). Hyperalgesia is defined as an increased sensitivity to normally painful stimuli. Examples of neuropathic pain include carpal tunnel syndrome, trigeminal neuralgia, post herpetic neuralgia, phantom limb pain, complex regional pain syndromes and the various peripheral neuropathies. Subsequent to nerve injury, there is increase in nerve traffic. Expression of sodium channels is altered significantly in response to injury thus leading to abnormal excitability in the sensory neurons. Nerve impulses arriving in the spinal cord stimulate the release of inflammatory protein Substance P. The presence of Substance P and other inflammatory proteins such as calcitonin gene-related peptide (CGRP) neurokinin A, vasoactive intestinal peptide removes magnesium induced inhibition and enables excitatory inflammatory proteins such as glutamate and aspartate to activate specialized spinal cord NMDA receptors. This results in magnification of all nerve traffic and pain stimuli that arrive in the spinal cord from the periphery.

In one study, monocytes/macrophages (ED-1), natural killer cells, T lymphocytes, and the pro-inflammatory cytokines tumor necrosis factor-alpha (TNF-alpha) and interleukin-6 (IL-6), were significantly produced in nerve-injured rats. Interestingly, ED-1-, TNF-alpha- and interLeukin-6-positive cells increased more markedly in allodynic rats than in non-allodynic ones. The magnitude of the inflammatory response was not related to the extent of damage to the nerve fibers because rats with complete transection of the nerves displayed much lower production of inflammatory cytokines than rats with partial transection of the nerve. This is a finding commonly observed in patients where a minor injury results in severe pain that is out of proportion to the injury. Referring again to the study, the authors determined that the considerable increase in monocytes/macrophages induced by a nerve injury results in a very high release of interleukin-6 and TNF-alpha. This may relate to the generation of touch allodynia/hyperalgesia, since there was a clear correlation between the number of ED-1 and interleukin-6-positive cells and the degree of allodynia.

Abnormal development of sensory-sympathetic connections follows nerve injury, and contributes to the hyperalgesia (abnormally severe pain) and allodynia (pain due to normally innocuous stimuli). These abnormal connections between sympathetic and sensory neurons arise in part due to sprouting of sympathetic axons. Studies have shown that sympathetic axons invade spinal cord dorsal root ganglia (DRG) following nerve injury, and activity in the resulting pericellular axonal baskets may underlie painful sympathetic-sensory coupling. Sympathetic sprouting into the DRG may be stimulated by neurotrophins such as nerve growth factor (NGF), brain derived neurotrophic factor (BDNF), neurotrophin-3 (NT-3) and neurotrophin 4/5 (NT-4/5).

In another study, animals exhibiting heat hyperalgesia as a sign of neuropathic pain seven days after loose ligation of the sciatic nerve exhibited a significant increase in the concentration of brain derived neurotrophic factor (BDNF) in their lumbar spinal dorsal horn. Administration of nerve growth factor to rodents has resulted in the rapid onset of hyperalgesia. In clinical trials with nerve growth factor for the treatment of Alzheimer disease and peripheral neuropathy, induction of pain has been the major adverse event.

In one study, the use of trkA-IgG, an inhibitor of nerve growth factor (NGF) reduced neuroma formation and neuropathic pain in rats with peripheral nerve injury. In another study, the systemic administration of anti-nerve growth factor (NGF) antibodies significantly reduced the severity of autotomy (self mutilating behavior induced by nerve damage) and prevented the spread of collateral sprouting from the saphenous nerve into the sciatic innervation territory.

Activity in sympathetic fibers is associated with excessive sweating, temperature instability of the extremities and can induce further activity in sensitized pain receptors and, therefore, enhance pain and allodynia (sympathetically maintained pain). This pathologic interaction acts via noradrenaline released from sympathetic terminals and newly expressed receptors on the afferent neuron membrane. Activation of motor nerves that travel from the spinal cord to the muscles results in excessive muscle tension. More inflammatory mediators are released which then excite additional pain receptors in muscles, tendons and joints generating more nerve traffic and increased muscle spasm. Persistent abnormal spinal reflex transmission due to local injury or even inappropriate postural habits may then result in a vicious circle between muscle hypertension and pain.

Separately, constant C-fiber nerve stimulation to transmission pathways in the spinal cord results in even more release of inflammatory mediators but this time within the spinal cord. The transcription factor, nuclear factor-kappa B (NF-kappaB), plays a pivotal role in regulating the production of inflammatory cytokines.

Inflammation causes increased production of the enzyme cyclooxygenase-2 (Cox-2), leading to the release of chemical mediators both in the area of injury and in the spinal cord. Widespread induction of Cox-2 expression in spinal cord neurons and in other regions of the central nervous system elevates inflammatory mediator prostaglandin E.sub.2 (PGE.sub.2) levels in the cerebrospinal fluid. The major inducer of central Cox-2 upregulation is inflammatory mediator interleukin-1.sup.beta. Din the CNS.sup.87. Basal levels of the enzyme phospholipase A.sub.2 activity in the CNS do not change with peripheral inflammation. The central nervous system response to pain can keep increasing even though the painful stimulus from the injured tissue remains steady. This “wind-up” phenomenon in deep dorsal neurons can dramatically increase the injured person's sensitivity to the pain. The neurotrophins are a family of growth promoting proteins that are essential for the generation and survival of nerve cells during development. Neurotrophins promote growth of small sensory neurons and stimulate the regeneration of damaged nerve fibers. They consist of four members, nerve growth factor (NGF), brain derived neurotrophic factor (BDNF), neurotrophin-3 (NT-3) and neurotrophin 4/5 (NT-4/5). Nerve growth factor and brain-derived neurotrophic factor modulate the activity of a sodium channel (NaN) that is preferentially expressed in pain signaling neurons that innervate the body (spinal cord dorsal root ganglion neurons) and face (trigeminal neurons).

Transection of a nerve fiber (axotomy) results in an increased production of inflammatory cytokines and induces marked changes in the expression of sodium channels within the sensory neurons. Following axotomy the density of slow (tetrodotoxin-resistant) sodium currents decrease and a rapidly repriming sodium current appears. The altered expression of sodium channels leads to abnormal excitability in the sensory neurons. Studies have shown that these changes in sodium channel expression following axotomy may be attributed at least in part to the loss of retrogradely transported nerve growth factor. In addition to effects on sodium channels, there is a large reduction in potassium current subtypes following nerve transection and neuroma formation. Studies have shown that direct application of nerve growth factor to the injured nerve can prevent these changes.

Reflex sympathetic dystrophy (RSD) syndrome, also called chronic regional pain syndrome (CRPS), has been recognized clinically for many years. It is most often initiated by trauma to a nerve, neural plexus, or soft tissue. Diagnostic criteria are the presence of regional pain and other sensory changes following a painful injury. The pain is associated with changes in skin color, skin temperature, abnormal sweating, tissue swelling. With time, tissue atrophy may occur as well as involuntary movements, muscle spasms, or pseudoparalysis. Like other organs with a blood supply, the bones also react to the disturbances in permeability caused by various inflammatory mediators. There is fluid accumulation in the bones and loss of bone density (osteoporosis). In addition, the inflammatory mediators accelerate the rate at which bone is broken down. The bone loss is further aggravated by decreased use of the affected body part due to pain. Complex regional pain syndrome, type I (reflex sympathetic dystrophy; CRPS-I/RSD) can spread from the initial site of presentation.

In one study of 27 CRPS-I/RSD patients who experienced a significant spread of pain, three patterns of spread were identified. Contiguous spread (CS) was noted in all 27 cases and was characterized by a gradual and significant enlargement of the area affected initially. Independent spread (IS) was noted in 19 patients (70%) and was characterized by the appearance of CRPS-I in a location that was distant and non-contiguous with the initial site (e.g. CRPS-I/RSD appearing first in a foot, then in a hand). Mirror-image spread (MS) was noted in four patients (15%) and was characterized by the appearance of symptoms on the opposite side in an area that closely matched in size and location the site of initial presentation. Only five patients (19%) suffered from CS alone; 70% also had IS, 11% also had MS, and one patient had all three kinds of spread.

In 1942 Paul Sudeck suggested that the signs and symptoms of RSD/CRPS including sympathetic hyperactivity might be provoked by an exaggerated inflammatory response to injury or operation of an extremity. His theory found no followers, as most doctors incorrectly believe that RSD/CRPS is solely initiated by a hyperactive sympathetic system. Recent research and studies including various clinical and experimental investigations now provide support to the theory of Paul Sudeck. As we now understand, soft tissue or nerve injury causes excitation of sensory nerve fibers. Reverse (antidromic) firing of these sensory nerves causes release of the inflammatory neuropeptides at the peripheral endings of these fibers. These neuropeptides may induce vasodilation, increase vascular permeability, attract other immune cells such as T helper cells and excite surrounding sensory nerve fibers—a phenomenon referred to as neurogenic inflammation.

While true inflammation is not present, this is a very good example of neuromodulatory molecules that are essentially non-inflammatory consistent with the “Deagle's Multilevel Pain Gate Model” where input stimuli above or below the subject gate structures increases pain signal transmission by modulating the pain gate at that level. At the level of the central nervous system, the increased input from peripheral pain receptors alters the central processing mechanisms. Sympathetic dysfunction, which often has been purported to play a pivotal role in RSD/CRPS, has been suggested to consist of an increased rate of outgoing (efferent) sympathetic nerve impulses towards the involved extremity induced by increased firing of the sensory nerves. However, the results of several experimental studies suggest that sympathetic dysfunction also consists of super sensitivity to catecholamines induced by nerve injury (autonomic denervation). Part of this occurs due to injured sensory nerves and immune cells developing receptors for the chemical transmitter norepinephrine and epinephrine (catecholamines), which are normally released by sympathetic nerves and also circulate in the blood. Stimulation of these receptors by locally released or circulating catecholamines produces sympathetic effects such as sweating, excessive hair growth and narrowing of blood vessels. In addition and under certain conditions, catecholamines may boost regional immune responses, through increased release of interleukin-1, tumor necrosis factor-alpha, and interleukin-8 production. In several studies, patients with RSD/CRPS showed a markedly increased level of the inflammatory peptide bradykinin as well as calcitonin gene-related peptide. The levels of bradykinin were four times as high as the controls. A few showed increased levels of the other inflammatory chemical mediators.

Two pain producing pathways have been identified: inflammatory stimuli induce the production of bradykinin, which stimulates the release of TNF-alpha. The TNF-alpha induces production of (i) interleukin-6 and interleukin-1b, which stimulate the production of cyclooxygenase products, and (ii) interLeuken-8, which stimulates production of sympathomimetics (sympathetic hyperalgesia). Abnormal development of sensory-sympathetic connections follow nerve injury, and contribute to the hyperalgesia (abnormally severe pain) and allodynia (pain due to normally innocuous stimuli). These abnormal connections between sympathetic and sensory neurons arise in part due to sprouting of sympathetic axons. This can be induced by neurotrophins such as nerve growth factor (NGF), brain derived neurotrophic factor (BDNF), neurotrophin-3 (NT-3) and neurotrophin 4/5 (NT-4/5).

The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be regarded as falling within the scope of the invention as defined by the claims that follow.

1. A method for treating a subject in need of treatment for persistent pain disorder facilitated by a biochemical mediator of inflammation, comprising:
selecting a target level of the multilevel pain gate selected from (1) Langerhans cellular junctional complex level, (2) dorsal root ganglionic level, (3) substantia gelatinosa level, (4) ascending spinothalamic tracts level, (5) subthalmic and thalamic nuclei level, (6) sensory receptive-motor parietal-temporal cortical level, and (7) the sensory-behavioral-motor frontal cortical level;
blocking receptors specific to pain gate facilitator molecules of said targeted pain gate level by administering to said subject an inhibitor specific to said biochemical mediator in an effective dose to increase pain gate inhibitory molecules.

2. The method according to claim 1, wherein said biochemical mediator of inflammation is selected from the group consisting of TNF-alpha, interluken-1, leukotriene, 5-lipoxygenase, nitric oxide, substance P, calcitonin gene-related peptide, vasoactive intestinal peptide, interluken-4, interluken-6, interluken-8, a kinin, serotonin, a matrix metallo-proteinase, inducible nitric oxide synthase, byproduct NO−, nitric oxide free radical, glial cells activated to release inflammatory molecules, and combinations thereof.

3. The method according to claim 1, wherein said inhibitor is chosen from the group consisting of TNF-alpha inhibitor, interleukin-1 receptor antagonist, leukotriene receptor antagonist, 5-lipoxygenase antagonist; nitric oxide antagonist; substance P antagoist; calcitonin gene-related peptide antagonist; vasoactive intestinal peptide antagonist; interleukin-4 antagonist; interleukin-6 antagonist; interleukin-8 antagonist, kinin antagonist, serotonin receptor antagonist; matrix metallo-proteinase antagonist; nitric oxide synthase antagonist; glial cell mediator antagonist; gene pain gate facilatory molecular inhibitor, gene pain gate inhibitory molecular upregulator, and combinations thereof.

4. The method according to claim 1, wherein said biochemical mediator of inflammation is selected from the group consisting of an ion channel mediator, a neurotrophin, and combinations thereof.

5. The method according to claim 4, wherein said ion channel mediator is an acid-sensing ion channel mediator (ASIC).

6. The method according to claim 5, wherein said acid sensing ion channel mediator is selected from the group consisting of ASIC1A receptor, ASIC3 receptor, and combinations thereof.

7. The method according to claim 4, wherein said neurotrophin is Nerve Growth Factor.

8. A method for treating a subject in need of treatment for persistent pain disorder facilitated by a biochemical mediator of inflammation, comprising:
selecting a target level of the multilevel pain gate selected from (1) Langerhans cellular junctional complex level, (2) dorsal root ganglionic level, (3) substantia gelatinosa level, (4) ascending spinothalamic tracts level, (5) subthalmic and thalamic nuclei level, (6) sensory receptive-motor parietal-temporal cortical level, and (7) the sensory-behavioral-motor frontal cortical level;
blocking receptors specific to pain gate facilitator molecules of said targeted pain gate level by administering to said subject an inhibitor specific to said biochemical mediator in an effective dose to increase pain gate inhibitory molecules, wherein said inhibitor is selected from the group consisting of a pharmaceutical, a nutraceutical, a gene insertion, a gene modulation, and combinations thereof.
